SEQUENCE LISTING (1 sequence)

SEQ ID NO: 1 - DNA, 30 bp, synthetic construct
  misc_feature 1..30: note = Synthetic
  source 1..30: mol_type = other DNA
gggggggggggacgatcgtcgggggggggg

(19) United States
(12) Patent Application Publication
(10) Pub. No.: US 2025/0034650 A1
(43) Pub. Date: Jan. 30, 2025

(54) TRANSCRIPTIONAL BIOMARKERS FOR RESPONSE TO INNATE IMMUNE ACTIVATORS

(71) Applicant: Checkmate Pharmaceuticals, Inc., Cambridge, MA (US)

(72) Inventors: Arthur M. KRIEG, Cambridge, MA (US); Hong LIU, Cambridge, MA (US)

(21) Appl. No.: 18/760,737

(22) Filed: Jul. 1, 2024

Related U.S. Application Data
(63) Continuation of application No. PCT/US2022/082627, filed on Dec. 30, 2022.
(60) Provisional application No. 63/296,098, filed on Jan. 3, 2022.

(51) Int. Cl. C12Q 1/6886; A61K 45/00

(52) U.S. Cl. CPC C12Q 1/6886; A61K 45/00; C12Q 2600/106; C12Q 2600/158

(57) ABSTRACT

Provided herein are methods and kits for diagnosing patients with cancer who are likely to respond to treatment with a TLR9 agonist or other innate immune activator or cancer immunotherapy based on detecting the level of expression of a vidutolimod core signature, and/or the high expression of ELF2 and/or low expression of a GABA B cell signature, and/or a low level of T_{reg} or macrophage/monocytes in the tumor. Also provided herein are methods of treating cancer, for example after determining the enrichment of the vidutolimod response signature, comprising administering CpG oligonucleotides formulated in a virus-like particle and, optionally, administering a checkpoint inhibitor such as an anti-PD-1 antibody, an anti-PD-L1 antibody.

### RELATED APPLICATIONS

This application is a continuation of International Patent Application No. PCT/US2022/082627, filed Dec. 30, 2022, which claims the benefit of U.S. Provisional Patent Application Ser. No. 63/296,098, filed Jan. 3, 2022, the entire disclosures of which are hereby incorporated herein by reference.

### INCORPORATION BY REFERENCE OF MATERIAL SUBMITTED ELECTRONICALLY

Incorporated by reference in its entirety is a computer-readable sequence listing submitted concurrently herewith and identified as follows: Filename: 755977_RGN9-009PCCON_ST26.xml; Size: 2,313 bytes; Created: Jul. 1, 2024.

### BACKGROUND OF THE INVENTION

Cancer immunotherapy is creating considerable excitement based, in large part, on the success of immune checkpoint blockade, such as inhibitors of the PD-1/PD-L1 pathway. Despite this excitement, most patients do not respond to PD-1 blockade, especially patients whose tumors lack an IFN signature. This is leading to evaluation of approaches designed to induce an IFN response such as innate immune activators including for example intratumoral (IT) delivery of agents capable of activating tumor-infiltrating plasmacytoid dendritic cells (pDC), thereby augmenting the tumor-specific T cell response.

Synthetic, unmethylated, CG-rich CpG oligodeoxynucleotides (ODN) mimic prokaryotic DNA and activate TLR9. Previously, PCT/US2015/067269 (published as WO/2016/109310) disclosed the use of CMP-001 (vidutolimod), a CpG-A ODN formulated in a VLP, in combination with checkpoint inhibitors (CPI). In patients with early stage melanoma receiving neoadjuvant treatment with PD-1 blockade, the response rate including pathologic cure was approximately doubled by the addition of IT vidutolimod (Davar, Diwakar, et al. “303 Phase II trial of neoadjuvant nivolumab (Nivo) and intra-tumoral (IT) CMP-001 in high-risk resectable melanoma (Nco-C-Nivo): final results.” (SITC 2020). In patients with metastatic melanoma refractory to PD-1 blockade, IT vidutolimod induced objective RECIST v1.1 response in 8/40 patients (20%) and the combination of IT vidutolimod with PD-1 blockade induced response in 23/98 patients (23%) (Kirkwood. John, et al. “950 Final analysis: phase 1b study investigating intratumoral injection of toll-like receptor 9 agonist vidutolimod±pembrolizumab in patients with PD-1 blockade-refractory melanoma.” (SITC 2021).

There remains a need to improve the efficacy of cancer therapy by identifying patients who are likely to respond to a particular anti-cancer therapy in advance of the therapy being administered. For example, it is well established in the field of cancer immunotherapy that PD-1 blockade is most effective in treating tumors that are “hot” or inflamed, and that treatment is much less likely to be effective in patients whose tumors are cold, or uninflamed at baseline, as judged by standard assays such as i) IHC for PD-L1 (combined positive score; CPS); ii) immunoscore; iii) TIDE (doi.org/10.1038/s41591-018-0136-1); iv) IFN18 signature (Ayers, M., et al., J Clin Invest., 2017, 127 (8): 2930-2940; and WO/2016/094377); and other assays known to those skilled in the art. These assays for predicting response to PD-1 blockade do not predict response to vidutolimod or other innate immune activators (Luke, Jason John, et al. “Abstract CT032: CMP-001 demonstrates improved response in noninflamed anti-PD-1 refractory melanoma and response is associated with serum CXCL10.” (SITC 2021): CT032-CT032.). Therefore, there is a need for assays to predict response to vidutolimod and other TLR9 agonists, or other TLR agonists (including for example, TLR4, TLR7, and TLR8 agonists), and/or other innate immune activators, ideally from baseline biopsies of patients' tumors or from liquid biopsies such as blood samples that contain tumor cells. Such assays would make it possible to greatly improve the response rate to vidutolimod and other TLR agonists or innate immune activators, by identifying the patients most likely to respond to such therapies in advance of treatment, and by providing other treatment options to patients whose baseline biopsies indicate that they are unlikely to respond to vidutolimod or other TLR agonists or innate immune activators.

### SUMMARY OF THE INVENTION

The present disclosure provides formulations, compositions and methods for treating cancer. As described herein, in one embodiment a method of identifying a subject that is likely to benefit from a cancer immunotherapy comprising an innate immune activator is provided, said method comprising: (i) obtaining a tumor sample from a subject with cancer; and (ii) detecting the expression level in the sample of at least 5 vidutolimod response core signature gene transcription products selected from the group consisting of SEC16B, AREG, COL7A1, SEC24D, CNIH2, MAPK15, TGFA, CUL3, SAR1B, USO1, TRAPPC6A, SEC24A, TMED2, SEC31A, BET1, GOLGA2, RAB1A, NAPA, LMAN1, TRAPPC3, PREB, SCFD1, SEC23A, CTSC, TRAPPC1, ANKRD28, SEC24B, MCFD2, CD59, TMED10, STX5, TRAPPC6B, PPP6R3, SEC13, TRAPPC9, CEP19, ARFGAP3, GOSR1, SEC23B, MIA3, and VAPA; wherein said detecting of an enriched level of expression identifies a subject that is likely to benefit from a cancer immunotherapy comprising an innate immune activator.

In another embodiment of the present disclosure, a method of identifying a subject that is likely to benefit from a cancer immunotherapy comprising an innate immune activator is provided, said method comprising: (i) obtaining a tumor sample from a subject with cancer; and (ii) confirming that the tumor is not “hot”; and iii) detecting the expression level in that sample of at least 5 vidutolimod response core signature gene transcription products selected from the group consisting of SEC16B, AREG, COL7A1, SEC24D, CNIH2, MAPK15, TGFA, CUL3, SAR1B, USO1, TRAPPC6A, SEC24A, TMED2, SEC31A, BET1, GOLGA2, RAB1A, NAPA, LMAN1, TRAPPC3, PREB, SCFD1, SEC23A, CTSC, TRAPPC1, ANKRD28, SEC24B, MCFD2, CD59, TMED10, STX5, TRAPPC6B, PPP6R3, SEC13, TRAPPC9, CEP19, ARFGAP3, GOSR1, SEC23B, MIA3, and VAPA; wherein said detecting of an enriched level of expression of at least 5 vidutolimod core signature gene transcription products in a tumor that is not “hot” identifies a subject that is likely to benefit from a cancer immunotherapy comprising an innate immune activator.

In still another embodiment, the present disclosure provides a method of identifying a subject that is likely to benefit from a cancer immunotherapy comprising an innate immune activator, said method comprising: (i) obtaining a tumor sample from a subject; (ii) detecting an elevated expression level in that sample of at least 5 vidutolimod response core signature gene transcription products selected from the group consisting of SEC16B, AREG, COL7A1, SEC24D, CNIH2, MAPK15, TGFA, CUL3, SAR1B, USO1, TRAPPC6A, SEC24A, TMED2, SEC31A, BET1, GOLGA2, RAB1A, NAPA, LMAN1, TRAPPC3, PREB, SCFD1, SEC23A, CTSC, TRAPPC1, ANKRD28, SEC24B, MCFD2, CD59, TMED10, STX5, TRAPPC6B, PPP6R3, SEC13, TRAPPC9, CEP19, ARFGAP3, GOSR1, SEC23B, MIA3, and VAPA; (iii) detecting the presence of a low macrophage and/or monocyte cell population; wherein said detecting identifies a subject that is likely to benefit from a cancer immunotherapy comprising an innate immune activator.

In yet another embodiment, the present disclosure provides a method of identifying a subject that is likely to benefit from a cancer immunotherapy comprising an innate immune activator, said method comprising: (i) obtaining a tumor sample from a subject; (ii) detecting an elevated expression level in that sample of at least 5 vidutolimod core signature gene transcription products selected from the group consisting of SEC16B, AREG, COL7A1, SEC24D, CNIH2, MAPK15, TGFA, CUL3, SAR1B, USO1, TRAPPC6A, SEC24A, TMED2, SEC31A, BET1, GOLGA2, RAB1A, NAPA, LMAN1, TRAPPC3, PREB, SCFD1, SEC23A, CTSC, TRAPPC1, ANKRD28, SEC24B, MCFD2, CD59, TMED10, STX5, TRAPPC6B, PPP6R3, SEC13, TRAPPC9, CEP19, ARFGAP3, GOSR1, SEC23B, MIA3, and VAPA; and (iii) detecting one or more of the following in the tumor sample: (a) the elevated expression of ELF2; and/or (b) the presence of a low T_{reg} cell population; and/or (c) low expression of a GABA B cell signature; wherein said detecting identifies a subject that is likely to benefit from a cancer immunotherapy comprising an innate immune activator.

In still another embodiment, the present disclosure provides a method of identifying a subject that is likely to benefit from a cancer therapy comprising an innate immune activator, comprising obtaining a tumor sample from the subject and detecting one or more of the following in the tumor sample: (a) the presence of a low macrophage and/or monocyte cell population; (b) high expression of ELF2; and/or (c) the presence of a low T_{reg} cell population; (d) low expression of a GABA B cell signature; wherein said detecting identifies a subject that is likely to benefit from a cancer therapy comprising an innate immune activator.

In some embodiments, the present disclosure provides a method of identifying a subject that is likely to benefit from a cancer therapy comprising an innate immune activator, wherein the innate immune activator is vidutolimod. In some embodiments, the present disclosure provides a method of identifying a subject that is likely to benefit from vidutolimod cancer therapy.

In another embodiment, the present disclosure provides a method of treating a subject afflicted with cancer with a cancer immunotherapy comprising an innate immune activator, said method comprising: (i) obtaining a tumor sample from a subject with cancer; and (ii) detecting the expression level in the sample of at least 5 vidutolimod response core signature gene transcription products selected from the group consisting of SEC16B, AREG, COL7A1, SEC24D, CNIH2, MAPK15, TGFA, CUL3, SAR1B, USO1, TRAPPC6A, SEC24A, TMED2, SEC31A, BET1, GOLGA2, RAB1A, NAPA, LMAN1, TRAPPC3, PREB, SCFD1, SEC23A, CTSC, TRAPPC1, ANKRD28, SEC24B, MCFD2, CD59, TMED10, STX5, TRAPPC6B, PPP6R3, SEC13, TRAPPC9, CEP19, ARFGAP3, GOSR1, SEC23B, MIA3, and VAPA, and, where an enriched level of expression is detected, and (iv) administering a cancer immunotherapy comprising an innate immune activator to the subject.

In still another embodiment, the present disclosure provides a method of treating a subject afflicted with cancer with a cancer immunotherapy comprising an innate immune activator, said method comprising: (i) obtaining a tumor sample from a subject with cancer; and (ii) confirming that the tumor is not “hot”; and (iii) detecting the expression level in that sample of at least 5 vidutolimod response core signature gene transcription products selected from the group consisting of SEC16B, AREG, COL7A1, SEC24D, CNIH2, MAPK15, TGFA, CUL3, SAR1B, USO1, TRAPPC6A, SEC24A, TMED2, SEC31A, BET1, GOLGA2, RAB1A, NAPA, LMAN1, TRAPPC3, PREB, SCFD1, SEC23A, CTSC, TRAPPC1, ANKRD28, SEC24B, MCFD2, CD59, TMED10, STX5, TRAPPC6B, PPP6R3, SEC13, TRAPPC9, CEP19, ARFGAP3, GOSR1, SEC23B, MIA3, and VAPA, and, where an enriched level of expression is detected, and (iv) administering a cancer immunotherapy comprising an innate immune activator to the subject.

In yet another embodiment, the present disclosure provides a method of treating a subject afflicted with cancer with a cancer immunotherapy comprising an innate immune activator, said method comprising: (i) obtaining a tumor sample from a subject; (ii) detecting an elevated expression level in that sample of at least 5 vidutolimod response core signature gene transcription products selected from the group consisting of SEC16B, AREG, COL7A1, SEC24D, CNIH2, MAPK15, TGFA, CUL3, SAR1B, USO1, TRAPPC6A, SEC24A, TMED2, SEC31A, BET1, GOLGA2, RAB1A, NAPA, LMAN1, TRAPPC3, PREB, SCFD1, SEC23A, CTSC, TRAPPC1, ANKRD28, SEC24B, MCFD2, CD59, TMED10, STX5, TRAPPC6B, PPP6R3, SEC13, TRAPPC9, CEP19, ARFGAP3, GOSR1, SEC23B, MIA3, and VAPA; and/or (iii) detecting the presence of a low macrophage and/or monocyte cell population; and (iv) where an enriched level of expression in (ii) is detected and/or where a low macrophage and/or monocyte cell population is detected, administering a cancer immunotherapy comprising an innate immune activator to the subject.

The present disclosure provides, in one embodiment, a method of treating a subject afflicted with cancer with a cancer immunotherapy comprising an innate immune activator, said method comprising: (i) obtaining a tumor sample from a subject; (ii) detecting an elevated expression level in that sample of at least 5 vidutolimod core signature gene transcription products selected from the group consisting of SEC16B, AREG, COL7A1, SEC24D, CNIH2, MAPK15, TGFA, CUL3, SAR1B, USO1, TRAPPC6A, SEC24A, TMED2, SEC31A, BET1, GOLGA2, RAB1A, NAPA, LMAN1, TRAPPC3, PREB, SCFD1, SEC23A, CTSC, TRAPPC1, ANKRD28, SEC24B, MCFD2, CD59, TMED10, STX5, TRAPPC6B, PPP6R3, SEC13, TRAPPC9, CEP19, ARFGAP3, GOSR1, SEC23B, MIA3, and VAPA; and (iii) detecting one or more of the following in the tumor sample: (a) the elevated expression of ELF2; and/or (b) the presence of a low T_{reg} cell population; and/or (c) low expression of a GABA B cell signature; and (iv) administering a cancer immunotherapy comprising an innate immune activator to the subject.

In still another embodiment, the present disclosure provides a method of treating a subject afflicted with cancer with a cancer immunotherapy comprising an innate immune activator, said method comprising obtaining a tumor sample from the subject and detecting one or more of the following in the tumor sample: (a) the presence of a low macrophage and/or monocyte cell population; (b) high expression of ELF2; (c) the presence of a low Treg cell population; and/or (d) low expression of a GABA B cell signature; and (iv) administering a cancer immunotherapy comprising an innate immune activator to the subject.

In some embodiments, the present disclosure provides a method of treating a subject afflicted with cancer with a cancer immunotherapy comprising an innate immune activator, wherein the innate immune activator is vidutolimod. In some embodiments, the present disclosure provides a method of treating a subject afflicted with cancer with vidutolimod cancer immunotherapy.

In some embodiments, an aforementioned method is provided wherein the innate immune activator is selected from the group consisting of a TLR agonist. In one embodiment, the TLR agonist is selected from the group consisting of a TLR9, TLR4, TLR7, and TLR8 agonist. In another embodiment, the TLR agonist is a TLR9 agonist. In still another embodiment, the TLR9 agonist is selected from the group consisting of A-class CpG DNA, C-class CpG DNA, E-class CpG DNA, A/E-class CpG DNA, P-class CpG DNA, and any combination thereof. In yet another embodiment, the TLR9 agonist is an A-class CpG DNA. In one embodiment, the A-class CpG DNA comprises the sequence GGGGGGGGGGGACGATCGTCGGGGGGGGGG (SEQ ID NO:1). In still other embodiments, the A-class CpG DNA is formulated as a virus-like particle (VLP). In another embodiment, the VLP comprises bacteriophage Qβ coat protein. In some embodiments, the present disclosure provides a method of treating a subject afflicted with cancer with a cancer immunotherapy comprising an innate immune activator, wherein the innate immune activator is an A-class CpG DNA comprising the sequence GGGGGGGGGGGACGATCGTCGGGGGGGGGG (SEQ ID NO:1) and is formulated as a VLP comprising the bacteriophage Qβ coat protein. In some embodiments, the innate immune activator recited in any of the aforementioned methods is vidutolimod.

In another embodiment, an aforementioned method is provided wherein the tumor is determined to be not “hot” on the basis of one or more of the following: (a) a TIDE score <−1; (b) low expression of an IFN-γ transcription signature comprising 5 or more of CD3D, IDO1, CIITA, CD3E, CCL5, GZMK, CD2, HLA-DRA, CXCL13, IL2RG, NKG7, HLA-E, LAG3, TAGAP, CXCL10, STAT1, GZMB, CXCR6 gene transcription products; (c) a low Immunoscore; and/or (d) a PD-L1 CPS<10.

In some embodiments, an aforementioned method is provided wherein the tumor is refractory to an inhibitor of a checkpoint selected from the group consisting of PD-1 and PD-L1. In yet other embodiments an aforementioned method is provided wherein the subject is a human afflicted with one or more cancerous tumors. In one embodiment, the cancerous tumor is a lymphoma or a cancerous tumor of a tissue or organ selected from the group consisting of skin, head and neck, esophagus, stomach, liver, colon, rectum, pancreas, lung, breast, cervix, ovary, kidney, bladder, prostate, thyroid, brain, muscle, and bone. In still another embodiment, the cancerous tumor is melanoma, NSCLC, and HNSCC. In yet another embodiment, the subject has received a therapy selected from the group consisting of radiotherapy, chemotherapy, immunotherapy, a therapy comprising a checkpoint inhibitor, surgery, hormone therapy.

In some embodiments an aforementioned method is provided wherein the detecting of a gene transcription product is selected from the group of techniques consisting of RNA sequencing (RNA-Seq), mRNA sequencing (mRNA-Seq), targeted RNA-Seq, and noncoding RNA-Seq, Nanostring, microarrays, or other hybridization based techniques.

In still other embodiments, the present disclosure provides an aforementioned method wherein the presence of 10, 15, 20, 25, 30, 35 or more vidutolimod core signature gene transcription products are detected. In some embodiments, the present disclosure provides an aforementioned method wherein the presence of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or 41 vidutolimod core signature gene transcription products are detected. In still other embodiments, the present disclosure provides an aforementioned method wherein the detected vidutolimod core signature gene transcription products have an enrichment score in the highest 80% of patient biopsies for the tumor type. In other embodiments, the present disclosure provides an aforementioned method wherein the detected vidutolimod core signature gene transcription products are expressed in one or more cells selected from the group consisting of plasmacytoid dendritic cells (pDC), plasma cells, mast cells, activated monocytes, macrophages, dendritic cells, and/or T cells.

In still other embodiments, the present disclosure provides an aforementioned method wherein the cancer immunotherapy is administered via intratumoral, peritumoral, systemic, intravenous, intraperitoneal, enteric, oral, intramuscular, subcutaneous, transmucosal, topical, intravesicular and/or transdermal routes. In one embodiment, the composition is administered intratumorally. In yet another embodiment, the method further comprises administering to the subject at least one dose of a composition comprising a checkpoint inhibitor (CPI). In still another embodiment, the CPI is an antibody or antigen-binding fragment thereof which binds specifically to an antigen selected from the group consisting of PD-1, PD-L1, LAG-3, TIM-3, and CTLA-4. In one embodiment, the CPI is an antibody or antigen-binding fragment thereof which binds specifically to PD-1. In yet another embodiment, the TLR9 agonist is vidutolimod and the CPI is a PD-1 inhibitor.

The present disclosure also provides kits. In one embodiment, a kit is provided comprising one or more compositions comprising reagents capable of detecting at least 5 vidutolimod core signature gene transcription products selected from the group consisting of SEC16B, AREG, COL7A1, SEC24D, CNIH2, MAPK15, TGFA, CUL3, SAR1B, USO1, TRAPPC6A, SEC24A, TMED2, SEC31A, BET1, GOLGA2, RAB1A, NAPA, LMAN1, TRAPPC3, PREB, SCFD1, SEC23A, CTSC, TRAPPC1, ANKRD28, SEC24B, MCFD2, CD59, TMED10, STX5, TRAPPC6B, PPP6R3, SEC13, TRAPPC9, CEP19, ARFGAP3, GOSR1, SEC23B, MIA3, and VAPA. In another embodiment, a kit is provided comprising one or more compositions comprising reagents capable of detecting one or more of: (a) the presence of a low macrophage and/or monocyte cell population; (b) high expression of ELF2; and/or (c) the presence of a low T_{reg} cell population; and/or (d) low expression of a GABA B cell signature.

### BRIEF DESCRIPTION OF THE DRAWINGS

FIG. 1 shows the results of Gene Set Enrichment Analysis (GSEA; www.gsea-msigdb.org/gsea/index.jsp; two groups comparison) performed on RNA Seq data from baseline “cold tumors” comparing subsequent Responder vs. PD in clinical trial NCT02680184 against all signatures in the molecular signatures database (MSigDB). Four gene signatures were identified that were enriched in Responders vs. PD (FDR<0.25, default cutoff),

FIG. 2 shows how Leading edge analysis was used to identify the core genes in the four signatures very highly enriched in vidutolimod responders who had baseline “cold” tumor biopsies with PD-1 refractory melanoma in clinical trial NCT02680184.

FIG. 3 shows a Venn diagram to illustrate that the core genes of “COPII_COATED_VESICLE_BUDDING” and “VESICLE_TARGETING_TO_FROM_OR_WITHIN_GOLGI” core genes are highly overlapping with each other, including 35 shared genes. In contrast, these two signatures share minimal overlap with the other 2 signatures initially associated with vidutolimod response.

FIG. 4A is an example of the plots showing that for the baseline biopsies of patients with PD-1 refractory melanoma in clinical trial NCT02680184 who had “cold” tumors, response to vidutolimod as monotherapy or in combination with pembrolizumab was associated with increased expression (“enrichment score”) of the Golgi core signatures (only 1 is shown for this example). Patient who would go on to have PD had the lowest enrichment scores, and patients who achieved SD had intermediate enrichment scores. FIG. 4B shows the same plot of enrichment scores for all of the baseline biopsies including those characterized as “cold”, “intermediate”, or “hot”. FIG. 4C shows that there is no relationship between subsequent response to vidutolimod and the enrichment score of the Golgi core signatures in the patients with baseline “hot” biopsies.

FIG. 5 shows that there is no significant difference in the overall enrichment of the Golgi core signatures between the patients with baseline “hot”, “intermediate”, or “cold” biopsies.

FIG. 6 shows the overall enrichment of the Golgi core signature by response status in the total baseline population (left hand panel) and by the baseline inflammatory status of the biopsies using the IFN18 signature. Since the patient data from the baseline “intermediate” biopsies had not been used for the original identification of the Golgi signature, these data provide independent confirmation of a significant association between the enrichment score for the Golgi signature and the probability of response to vidutolimod+pembrolizumab therapy.

FIG. 7 shows another independent dataset confirming a significant association between the enrichment score for the Golgi signature and the probability of tumor shrinkage in patients with PD-1 refractory cancer (in this case, NSCLC in clinical trial NCT03438318) to vidutolimod+PD-1 blockade (in this case with anti-PD-L1 atezolizumab) therapy.

FIG. 8 shows from public datasets that the enrichment score of the Golgi core signature is NOT associated with response to nor induced by PD-1 or CTLA-4 blockade.

FIG. 9 shows using public single cell RNA Seq datasets from various tumors (in this case from a melanoma) that the Golgi signature is most highly expressed within tumor-associated plasma cells, followed by pDC, malignant cell, macrophage, and immature DC.

FIG. 10 shows using a public single cell RNA Seq dataset from purified pDC activated in vitro that the Golgi signature is most highly expressed within the P1 pDC subset, which was also identified as the major subset producing type I IFN.

FIG. 11 shows that within the TCGA database of RNA Seq from thousands of tumors, the Golgi signature is highly expressed across most tumor types.

FIG. 12 shows using a public RNA Seq dataset from irradiated and normal tissue that there is a trend for higher enrichment of the Golgi core signature in irradiated normal or malignant cells.

FIG. 13 shows the results of an analysis using Qlattice, an AI tool, identifying ELF2 as a potential “partner” gene for the Golgi signature using a novel algorithm that may better predict response to vidutolimod (and other innate immune activators or TLR agonists) from gene expression data of baseline tumor biopsies.

FIG. 14 shows that the expression of ELF2 demonstrates an independent association with response to vidutolimod regardless of the baseline tumor inflammation status.

FIG. 15 shows that for our RNA Seq dataset of patients with PD-1 refractory melanoma, BRAF V600E mutation status is associated with the published IFN18 signature, but not with Golgi signatures, and that there is no association of Golgi signature expression with prior Braf inhibitor treatment.

FIG. 16 reveals that the enrichment of the Golgi core signature is not associated with baseline LDH or liver metastases, which are known adverse prognostic factors for the response of melanoma to immunotherapy.

FIG. 17 reveals that the enrichment of the Golgi core signature is not associated with baseline tumor burden, another known adverse prognostic factors for the response of melanoma to immunotherapy.

FIG. 18 shows that baseline “hot” biopsies with high signatures for macrophage/monocytes are unlikely to respond to vidutolimod+pembrolizumab.

FIG. 19 shows that low expression of macrophage/monocyte-expressed genes including TLR8, TGF-b, TNFR, C1QC, or PD-L2 predicts clinical response in baseline “hot” tumor biopsies.

FIG. 20 shows that low expression of TNF-α or TLR9 predicts clinical response in cold but not hot tumor biopsies.

FIG. 21 among all baseline biopsies, those with high signatures for T_{reg} are unlikely to respond to vidutolimod+pembrolizumab.

FIG. 22 shows that among the baseline hot biopsies, those with high signatures for GABA B cell activation are unlikely to respond to vidutolimod+pembrolizumab.

### DETAILED DESCRIPTION OF THE INVENTION

The present disclosure provides biomarkers for response to cancer therapy including, for example, a cancer immunotherapy. As used herein, the term “cancer immunotherapy” refers to a cancer treatment providing benefit primarily or largely through effects on immune function, as opposed to direct effects on tumor cells. Cancer immunotherapies include i) checkpoint inhibitors that essentially function to “remove the brakes” on the immune system; and ii) innate immune activators that function to stimulate innate and adaptive immunity leading to anti-tumor immunity. As used herein, innate immune activators relevant for cancer immunotherapy include for example various Toll-like receptors (TLR), especially TLR3, TLR4, TLR7, TLR8, and TLR9, RIG-I-like receptors (RLR), cGAS/STING, cytokines, interferons, and oncolytic viruses.

As used herein, “biomarker” or “biomarkers” refers to the presence or absence in a sample of: a gene product (e.g., a transcript or a protein), a cell type or a cell type population, and/or a protein, cytokine or hormone. The presence or absence, either individually or collectively, of the biomarkers described herein are used to determine or otherwise identify a patient that is likely to benefit from a cancer treatment that includes an innate immune activator such as a TLR9 agonist.

In some embodiments the cancer immunotherapy may include a TLR9 agonist in combination with a checkpoint inhibitor, such as an antibody to PD-1. Exemplary TLR9 agonists and checkpoint inhibitors are provided herein. Additionally, as will be appreciated by those of skill in the art, the signatures and methods described herein also encompass cancer treatments that include other TLR agonists, including TLR3, TLR4, TLR7, and TLR8 agonists.

A patient that is likely to benefit from a cancer therapy as used herein is a patient that will demonstrate an alleviation of one or more cancer symptom including, for example, by the induction of an anti-tumor response. An anti-tumor response, when referring to a cancer patient treated with a therapeutic agent, TLR9 agonist (optionally in combination with a checkpoint inhibitor, such as an antibody to PD-1), means at least one positive therapeutic effect, such as for example, reduced number of cancer cells, reduced tumor size, reduced rate of cancer cell infiltration into peripheral organs, reduced rate of tumor metastasis or tumor growth, or progression free survival. Positive therapeutic effects in cancer can be measured in a number of ways (See, W. A. Weber, J. Null. Med. 50:1S-10S (2009); Eisenhauer et al., supra). In some embodiments, an anti-tumor response to a PD-1 antagonist is assessed using RECIST 1.1 criteria, bidimensional irRC or unidimensional irRC. In some embodiments, an antitumor response is any of SD (stable disease), PR (partial response), CR (complete response), PFS (progression-free survival), DFS (disease-free survival) or OS (overall survival). In some embodiments, a transcriptional signature of the present disclosure predicts whether a subject with a solid tumor is likely to achieve a PR or a CR.

Transcriptional signatures and signature or gene biomarkers derived using the methods described herein may be useful to identify cancer patients who are most likely to achieve a clinical benefit from treatment with a TLR9 agonist (optionally in combination with one or more checkpoint inhibitors, such as an antibody to PD-1). This utility supports the use of such biomarkers in a variety of research and commercial applications, including but not limited to, clinical trials of innate immune activators such as TLR9 agonists (optionally in combination with a checkpoint inhibitor, such as an antibody to PD-1) in which patients are selected on the basis of whether they test positive or negative for a gene signature or gene biomarker, diagnostic methods and products for determining a patient's gene signature score or for classifying a patient as positive or negative for a gene signature and/or gene biomarker, personalized treatment methods which involve tailoring a patient's drug therapy based on the patient's gene signature score or biomarker status, as well as pharmaceutical compositions and drug products comprising a TLR9 agonist (optionally in combination with a checkpoint inhibitor, such as an antibody to PD-1) for use in treating patients who test positive for a gene signature biomarker.

The utility of any of the research and commercial applications claimed herein does not require that 100% of the patients who test positive for a gene signature biomarker achieve an anti-tumor response to an innate immune activator such as a TLR9 agonist (optionally in combination with a checkpoint inhibitor, such as an antibody to PD-1); nor does it require a diagnostic method or kit to have a specific degree of specificity or sensitivity in determining the presence or absence of a biomarker in every subject, nor does it require that a diagnostic method claimed herein be 100% accurate in predicting for every subject whether the subject is likely to have a beneficial response to an innate immune activator such as a TLR9 agonist (optionally in combination with a checkpoint inhibitor, such as an antibody to PD-1). Thus, the present disclosure herein provides that the terms “determine”, “determining” and “predicting” should not be interpreted as requiring a definite or certain result; instead these terms should be construed as meaning either that a claimed method provides an accurate result for at least the majority of subjects or that the result or prediction for any given subject is more likely to be correct than incorrect.

Biomarkers Associated with Response to TLR9 Agonist Cancer Therapy

The present disclosure provides biomarkers for response to cancer therapy. The present disclosure further provides, for example once a patient who is likely to benefit from cancer therapy is identified, formulations, compositions and methods for promoting immune activation and reducing immune inhibition, thus metaphorically both “stepping on the gas” and “releasing the brakes” of the immune system, to treat cancer. The disclosure can be used, for example, to convert “cold” (uninflamed, treatment-resistant or -refractory) cancers or tumors to “hot” (or inflamed) ones amenable to treatment, including treatment with checkpoint inhibition. For example, TLR9 agonists as described herein are used to convert so called “cold tumors”—i.e. tumors with a low degree of infiltration by activated immune cells—or intermediate tumors into “hot tumors”, which are immunogenic tumors, i.e., with an intermediate or high degree of activated immune cell infiltration. TLR9 agonists also can be used in the treatment of patients with “hot” tumors, either by themselves or in combination with for example CPI such as PD-(L) 1 blockers.

The concept of “cold” and “hot” tumors is well known to the person skilled in the art. Cold tumors are often enriched in immunosuppressive cytokines and may have high numbers of T_{reg} cells and/or myeloid-derived suppressor cells (MDSC). Cold tumors usually have relatively fewer activated T_{H1} cells, NK cells and CD8⁺ T cells and fewer functional antigen-presenting cells (APC) (for example dendritic cells/DC). In contrast, hot tumors are relatively enriched in T_{H1}-type chemokines and have relatively higher numbers of activated effector immune cells (T_{H1} cells, NK cells and CD8⁺ T cells) and/or higher numbers of functional DC.

The degree of immune cell infiltration can for example be measured by the so called “Immunoscore”, which is used to predict clinical outcome in patients with cancer. The consensus Immunoscore is a scoring system to summarize the density of CD3⁺ and CD8⁺ T cells within the tumor and its invasive margin. For example, the Immunoscore can be classified as low, intermediate and high depending on the CD3⁺/CD8⁺ T cell density, whereas a 0-25% density is preferably scored as low, a 25-70% density is preferably scored as intermediate and a 70-100% density is preferably scored as high (Pages F. et al. (2018) Lancet 391(10135):2128-2139) in a reference study. Cold tumors are defined as having a low degree of immune cell infiltration, i.e., preferably have a low Immunoscore. Hot tumors may be defined as having an intermediate or high degree of immune cell infiltration, i.e., preferably have an intermediate or high Immunoscore.

Some tumor types often belong to the type of hot tumors even before treatment, for example melanoma. These hot tumors respond more frequently than cold tumors to checkpoint inhibitors. Cold tumors usually do not respond well to checkpoint inhibitors. The present disclosure provides that a TLR9 agonist can improve such responsiveness by increasing the infiltration of immune cells into the tumor and thereby positively influencing the tumor microenvironment. Patients with cold tumors may therefore especially benefit from the treatment with a TLR9 agonist. The tumor may as a result show better responsiveness to checkpoint inhibitors. Cold tumors may thereby be converted into hot tumors.

Tumor infiltrating immune cells, the presence of which may indicate a hot tumor, include but are not limited to, T_{H1} cells, natural killer (NK) cells, CD8⁺ T cells, and/or dendritic cells (DC), especially when activated. Likewise, the presence of pro-inflammatory cytokines such as interferon, IL-12, IFN-g, and/or type I IFN are also indicative of a hot tumor.

TIDE is an algorithm developed to estimate the level of immune dysfunction in a tumor as another approach to functionally distinguish tumors that are likely to respond to CPI from those that are not based in part on the level of tumor inflammation.

WO/2016/094377 describes one of several approaches to distinguishing hot/inflamed from cold/uninflamed tumors, in this case using transcriptional profiling to determine the level of the RNA transcripts for “IFN18” genes CD3D, IDO1, CIITA, CD3E, CCL5, GZMK, CD2, HLA-DRA, CXCL13, IL2RG, NKG7, HLA-E, LAG3, TAGAP, CXCL10, STAT1, GZMB, CXCR6.

A transcriptional signature (i.e., a biomarker signature associated with response to TLR9 agonist cancer therapy) is determined in a sample of tumor tissue removed from a subject. The tumor may be primary or recurrent, and may be of any type (as described above), any stage (e.g., Stage I, II, III, or IV or an equivalent of other staging system), and/or histology. The subject may be of any age, gender, treatment history and/or extent and duration of remission. The tumor sample can be obtained by a variety of procedures including, but not limited to, surgical excision, aspiration or biopsy. In some embodiments the tumor sample may be obtained from a “liquid biopsy” or blood sample from which tumor cells can be detected and studied. The tissue sample may be sectioned and assayed as a fresh specimen; alternatively, the tissue sample may be frozen for further sectioning. In some embodiments, the tissue sample is preserved by fixing and embedding in paraffin or the like.

Once a suitable sample of tumor tissue has been obtained, it is analyzed in some embodiments to quantitate the RNA expression level for each of, or 1 or more of, or 5 or more of etc., the vidutolimod response core signature genes or other genes and signatures provided herein, or for a gene signature derived therefrom. In some embodiments, the sample is analyzed to quantitate the RNA expression level for 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or 41 of the vidutolimod response core signature genes or other genes and signatures provided herein, or for a gene signature derived therefrom. The phrase “determine the RNA expression level of a gene” as used herein refers to detecting and quantifying RNA transcribed from that gene. The term “RNA transcript” includes mRNA transcribed from the gene, and/or specific spliced variants thereof and/or fragments of such mRNA and spliced variants.

A person skilled in the art will appreciate that a number of methods can be used to isolate RNA from the tissue sample for analysis. For example, RNA may be isolated from frozen tissue samples by homogenization in guanidinium isothiocyanate and acid phenol-chloroform extraction. Commercial kits are available for isolating RNA from FFPE samples and many companies provide this service on tumor biopsies. If the tumor sample is a formalin-fixed paraffin-embedded (FFPE) tissue section on a glass slide, it is possible to perform gene expression analysis on whole cell lysates rather than on isolated total RNA.

Persons skilled in the art are also aware of several methods useful for detecting and quantifying the level of RNA transcripts within the isolated RNA or whole cell lysates. Quantitative detection methods include, but are not limited to, arrays (i.e., microarrays), quantitative real time PCR (RT-PCR), multiplex assays, nuclease protection assays, and Northern blot analyses. Generally, such methods employ labeled probes that are complementary to a portion of each transcript to be detected. Probes for use in these methods can be readily designed based on the known sequences of the genes and the transcripts expressed thereby. In some embodiments, a probe for detecting a transcript of a vidutolimod response core signature gene provided herein is designed to specifically hybridize to the target region for that gene. Suitable labels for the probes are well-known and include, e.g., fluorescent, chemilumnescent and radioactive labels.

In some embodiments, assaying a tumor sample for expression of the vidutolimod response core signature genes provided herein, or transcriptional signatures derived therefrom, employs detection and quantification of RNA levels in real-time using nucleic acid sequence based amplification (NASBA) combined with molecular beacon detection molecules. NASBA is described, e.g., in Compton J., Nature 350 (6313): 91-92 (1991). NASBA is a single-step isothermal RNA-specific amplification method. In other embodiments, the assay format is a flap endonuclease-based format, such as the Invader™ assay (Third Wave Technologies). In yet other embodiments, the assay format employs direct mRNA capture with branched DNA (QuantiGene™, Panomics) or Hybrid Capture™ (Digene).

One example of an array technology suitable for use in measuring expression of the genes in gene expression platform of the invention is the ArrayPlate™ assay technology sold by HTG Molecular, Tucson Arizona, and described in Martel, R. R., et al, Assay and Drug Development Technologies 1 (1): 61-71, 2002.

In another embodiment, one assay method to measure transcript abundance for the vidutolimod response core signature genes provided herein utilizes the nCounter® Analysis System marketed by NanoString® Technologies (Seattle, Washington USA). This system, which is described by Geiss et al., Nature Biotechnol. 2 (3): 317-325 (2008), utilizes a pair of probes, namely, a capture probe and a reporter probe, each comprising a 35- to 50-base sequence complementary to the transcript to be detected. The capture probe additionally includes a short common sequence coupled to an immobilization tag, e.g. an affinity tag that allows the complex to be immobilized for data collection. The reporter probe additionally includes a detectable signal or label, e.g. is coupled to a color-coded tag. Following hybridization, excess probes are removed from the sample, and hybridized probe/target complexes are aligned and immobilized via the affinity or other tag in a cartridge. The samples are then analyzed, for example using a digital analyzer or other processor adapted for this purpose. Generally, the color-coded tag on each transcript is counted and tabulated for each target transcript to yield the expression level of each transcript in the sample. This system allows measuring the expression of hundreds of unique gene transcripts in a single multiplex assay using capture and reporter probes designed by NanoString.

In measuring expression of the vidutolimod response core signature genes and other genes provided herein, the absolute expression of each of the genes in a tumor sample is in some embodiments compared to a control such as one or more housekeeping genes or constitutively expressed genes for the same tumor sample. By this means the expression level for the biomarker signature or genes is normalized within each sample, making it possible to compare the relative level of expression of the biomarker signature or gene across samples, thereby defining an enrichment score for a particular sample or set of samples compared to the average expression level for a particular population or dataset. To increase the sensitivity of the comparison the expression level values are preferably transformed in any of a number of ways. Thus, as used herein, the phrase “detecting an enriched level of expression . . . ” when referring to applications for distinguishing patients with cancer who are likely to respond to treatment with an innate immune activator, means showing a relative magnitude of RNA expression of a biomarker signature or gene that is greater than the magnitude in patients who have a low probability of responding to treatment with an innate immune activator, such as a TLR9 agonist. As will be appreciated by those of skill in the art, the cutoff for the enrichment score used as an assay to select patients to be treated with a therapy will be based on the sensitivity and specificity of the biomarker for predicting response in a particular patient population, and will be determined using standard methods in the field of biomarkers. Ideally, the cutoff for the enrichment score includes all of the patients who would respond to the treatment and excludes all of the patients who would not respond. The threshold may thus depend on many factors that are considered to provide the greatest chance for successful tumor therapy to an individual patient depending on their personal characteristics and on other available treatment options. For example, the cutoff for enrichment score can be determined based on the average expression levels in a data set for at least 5 vidutolimod response core signature gene transcription products (see, e.g. Jiang et al. Nature Medicine, 24 (2018): 1550-1558). In some embodiments, the cutoff for enrichment score is determined using any of the statistical methods described in Ayers et al. (WO/2016/094377), which is incorporated by reference herein in its entirety.

The enrichment score may be used in combination with other prognostic factors or biomarkers to make this prediction. Non-limiting examples of other prognostic factors or biomarkers include: (a) presence/absence of a low macrophage and/or monocyte cell population; (b) level of expression of ELF2: (c) presence/absence of a low T_{reg} cell population; and/or (d) level of expression of a GABA B cell signature.

For the purpose of selecting patients to be treated with a cancer therapy, the desired level of enrichment will be set in a way that normalizes the signature expression to housekeeping or other RNAs selected as internal controls for the purpose of standardizing the enrichment scores across different biopsies and datasets using standard methods well known to those expert in the art. Different enrichment scores may be used to select patients for treatment depending on the stage and location of the tumor, on the type of tumor, on the history of other treatments, and on the presence or absence of other biomarker signatures and genes. In some embodiments “partner” genes or signatures are mathematically combined with a biomarker gene or signature to provide a greater level of discrimination in selecting patients with the highest probability of response to a treatment, without excluding patients who would be responders. Considering all of these factors, an enriched level of expression for the purpose of predicting responders may be greater than the level of expression in the patients with the lowest 10% of expression (i.e., the enrichment score cutoff may include 90% of the patients in a particular population), or the lowest 20% of expression, or the lowest 30%, or the lowest 40%, or in some embodiments an enriched level of expression is greater than the average level of expression in the total population. In some embodiments the enrichment score used to select patients for treatment includes only the highest 10% of expression, or the highest 20%, or the highest 30%, or the highest 40%, or intermediate levels between any of these values.

Likewise, where other signatures or genes are detected or measured, measurements of cell population abundance, gene expression, or other marker are made based on well-known standards or by comparison to control samples. For example, as used herein, the phrase “elevated expression of ELF2” means in a similar fashion to the use of enrichment score above, a level of expression that is associated with a higher probability of response to an innate immune activator such as a TLR9 agonist compared to patients with tumors containing a lower relative level of ELF2 RNA compared to control genes. Some biomarker signatures and genes provided herein are associated with resistance to treatment with an innate immune activator or TLR9 agonist, and so with these biomarkers, selection for treatment is based on detecting a reduced level of expression or enrichment compared to the total population, in a manner inverse to that described above for biomarker signatures and genes that are positively associated with response.

Signatures for the immune cells described herein are contained within the Molecular Signatures Database (MSigDB) and for example include REACTOME_GABA_B_RECEPTOR_ACTIVATION for a GABA B cell signature, and immune cell signatures such as T_cell_regulatory_(T_{regs})_CIBERSORT for T_{reg}, Macrophage/Monocyte_MCPCOUNTER for macrophage/monocyte. The phrases “low T_{reg} cell population”, “low expression of a GABA B cell signature”, “low expression of a macrophage/monocyte signature, or low expression of any of TLR8, TLR9, TGFB2, TNFA, TNFRSF1A, TNFRSF1B, C1QC, and/or PD-L1, as used herein refer to a relative level of expression of any one or more of these signatures and/or genes that has been shown to be associated with an increased probability of response to an innate immune activator such as a TLR9 agonist as compared to patients with a higher level of expression within a particular patient population as described herein.

Raw expression values of the clinical response genes in a gene expression platform described herein may be normalized by any of the following: quantile normalization to a common reference distribution, by the mean RNA levels of a set of housekeeping genes, by global normalization relying on percentile, e.g., 75th percentile, or other biologically and clinically relevant normalization approaches known to those skilled in the art.

For example, the expression level of each clinical response gene can be normalized by the average RNA expression level of all of the genes in the gene expression platform, or by the average expression level of a set of normalization genes, e.g., housekeeping genes. Thus, in one embodiment, the genes in a gene expression platform are represented by a set of probes, and the RNA expression level of each of the genes is normalized by the mean or median expression level across all of the represented genes, i.e., across all clinical response and normalization genes in a gene expression platform described herein In a specific embodiment, the normalization is carried out by dividing the median or mean level of RNA expression of all of the genes in the gene expression platform. In another embodiment, the RNA expression levels of the clinical response genes are normalized by the mean or median level of expression of a set of normalization genes. In a specific embodiment, the normalization genes comprise housekeeping genes. In another specific embodiment, the normalization of a measured RNA expression level for a clinical response gene is accomplished by dividing the measured level by the median or mean expression level of the normalization genes.

When comparing a subject's tumor sample with a standard or control, the expression value of a particular gene in the sample is compared to the expression value of that gene in the standard or control. In some embodiments, for each gene in a gene signature of the invention, the log(10) ratio is created for the expression value in the individual sample relative to the standard or control. A score for a gene signature is calculated by determining the mean log(10) ratio of the genes in the signature. If the gene signature score for the test sample is equal to or greater than a pre-determined threshold for that gene signature, then the sample is considered to be positive for the gene signature biomarker. The pre-determined threshold may also be the mean, median, or a percentile of scores for that gene signature in a collection of samples or a pooled sample used as a standard or control.

The signatures derived herein were defined in patients who were refractory to PD-1 blockade. That is, patients who originally had tumor signatures predicting response to PD-1 blockade would usually have responded to the CPI and would not be candidates for a clinical trial with vidutolimod. When a patient who is naïve to PD-1 blockade is being considered for treatment with a combination therapy of an innate immune activator in combination with a checkpoint inhibitor, separate biomarkers may be used to predict the probability that the patient will respond to the checkpoint inhibitor alone, and to predict response to the innate immune activator. Patients who have biomarkers predicting response to the checkpoint inhibitor (such as TIDE, IFN18, CPS, Immunoscore, etc) may be selected for treatment with the combination if they show enrichment of one or more of the biomarkers described herein for predicting response to the innate immune activator, even if their level of enrichment for the biomarker for response to the innate immune activator is below the threshold that would otherwise be used if the patient did not have a signature predicting response to the PD-1 blockade (i.e., if the patient had a “cold” tumor). It is believed that addition of an innate immune activator to a checkpoint inhibitor may improve the duration and depth of response compared to treatment with the checkpoint inhibitor alone, and the biomarker strategies described herein can help in the selection of patients most like to benefit from such combinations.

It will be recognized by those skilled in the art that other differential expression values, besides log(10) ratio, may be used for calculating a signature score, as long as the value represents an objective measurement of transcript abundance of the genes. Examples include, but are not limited to: xdev, error-weighted log (ratio), and mean subtracted log (intensity).

Assaying tumor samples for expression of the genes in a gene expression platform or gene signature described herein may be performed using a kit that has been specially designed for this purpose. In one embodiment, the kit comprises a set of oligonucleotide probes capable of hybridizing to the set of target transcripts such as the vidutolimod response core signature gene transcription products described herein. The set of oligonucleotide probes may comprise an ordered array of oligonucleotides on a solid surface, such as a microchip, silica beads (such as BeadArray technology from Illumina, San Diego, CA), or a glass slide (see, e.g., WO 98/20020 and WO 98/20019). In some embodiments, the oligonucleotide probes are provided in one or more compositions in liquid or dried form. Kits may also include, in some embodiments, reagents for RNA sequencing (RNA-Seq), mRNA sequencing (mRNA-Seq), targeted RNA-Seq, and noncoding RNA-Seq, Nanostring, microarrays, or other hybridization based techniques. For example kits may comprise, in some embodiments, reagents that are capable of detecting the presence of, or determine the expression level of, one or more of target transcripts such as the vidutolimod response core signature gene transcription products described herein.

Kits of the present disclosure may also contain other reagents such as hybridization buffer and reagents to detect when hybridization with a specific target molecule has occurred. Detection reagents may include biotin- or fluorescent-tagged oligonucleotides and/or an enzyme-labeled antibody and one or more substrates that generate a detectable signal when acted on by the enzyme. It will be understood by the skilled artisan that the set of oligonucleotides and reagents for performing the assay will be provided in separate receptacles placed in the kit container if appropriate to preserve biological or chemical activity and enable proper use in the assay.

### Transcriptional Biomarkers

As described herein, the present disclosure provides transcriptional markers (e.g., mRNA transcripts) that can be used to determine whether a patient will benefit from, for example, a TLR9-based cancer therapy. In some embodiments, the transcriptional markers are related to genes involved in ER-to-Golgi transport and vesicle transport. In some embodiments these genes include some or all of SEC16B, AREG, COL7A1, SEC24D, CNIH2, MAPK15, TGFA, CUL3, SAR1B, USO1, TRAPPC6A, SEC24A, TMED2, SEC31A, BET1, GOLGA2, RAB1A, NAPA, LMAN1, TRAPPC3, PREB, SCFD1, SEC23A, CTSC, TRAPPC1, ANKRD28, SEC24B, MCFD2, CD59, TMED10, STX5, TRAPPC6B, PPP6R3, SEC13, TRAPPC9, CEP19, ARFGAP3, GOSR1, SEC23B, MIA3, VAPA, referred to herein as the “vidutolimod response core signature” or the “vidutolimod core signature.”

Like the closely related endosomal innate immune receptors TLR3, TLR7, and TLR8, TLR9 is produced in and resides predominantly in the endoplasmic reticulum (ER) in resting cells (Chockalingam, A., et al., Immunol. And Cell Biology, 2009, 87, 209-217). TLR9 redistributes to early endosomes upon stimulation with CpG-DNA. Endolysosomal localization is critical for TLR9 response to CpG DNA, as CpG-A DNAs that maximally induce IFN-α production accumulate in early endosomes, whereas CpG-B and CpG-C DNAs with phosphorothioate backbones that induce stronger inflammatory cytokines and immune cell maturation accumulate and activate TLR9 in late endosomes and/or in lysosomes. A pool of TLR9 is believed to constitutively traffic from the ER through the Golgi complex and to reside in endolysosomes, where this pool of TLR9 is thought to be involved in signaling (Chockalingham et al). The protein Unc93b1 is required for TLR9 trafficking from the ER through the Golgi to the endolysosome (reviewed in Ewald, S. E., and Barton, G. M., Current Opin. Immunol., 2011, 23:3-9). The ectodomains of TLR9 (and TLR7) are cleaved in the endolysosome, perhaps by cathepsins such as cathepsin K, and cleavage of the TLR9 ectodomain appears to be essential for responses to CpG DNA (Ewald, S. E., et al., Nature Letters, doi.10.1038/nature07405; Ewald, S. E., and Barton, G. M., Current Opin. Immunol., 2011, 23:3-9). Furthermore, transit of TLR9 through the Golgi complex is reported to occur in response to CpG DNA and to be required for optimal signaling (Chockalingham et al).

Confocal microscopy reveals that HMGB1 pre-associates with TLR9 and co-localizes with markers of the ER, the ERGIC, and the Golgi in quiescent cells (Ivanov, S., et al., Blood, 2007, 110:6, 1970-1981). Upon stimulation with CpG oligodeoxynucleotides (CpG-ODN), HMGB1 and TLR9 colocalize with the early endosomal marker EEA1. Ablation or depletion of HMGB1 impaired redistribution of TLR9 to early endosomes in response to CpG-ODN. As a consequence, HMGB1-deficient cells exhibited substantially decreased responses to CpG-ODN (Ivanov et al). Rab proteins are key-regulators of intracellular membrane trafficking. For example, Rab7 regulates the transport to late endosomes and lysosomes, and is important for lysosomal and phagosomal biogenesis, and for maturation of late autophagic vacuoles, while Rab7b controls vesicular trafficking from endosomes to the trans Golgi network (TGN) (Bucci, C., et al., Commun. And Integrative Biology, 2010, 3:5, 401-404). RAB7 overexpression in mouse B cells upregulates the activity of TRAF6, enhancing NF-kB activation (Yan, Hui, et al., The Journal of Immunology, 204.5 (2020): 1146-1157). High level expression of Rab7b downregulates the TLR4 and TLR9-mediated inflammatory response whereas depletion leads to upregulation of these responses (Bucci, C., et al., Commun. And Integrative Biology, 2010, 3:5, 401-404). Proprotein convertase 1/3 (PC1/3) is another key regulator of TLR4 and TLR9 dependent signaling, and rapidly co-localizes with TLR9 in CpG-ODN-containing endosomes following CpG stimulation, preventing TLR9 clustering in multivesicular bodies (MVB) and co-localization with Rab7 (Duhamel, M., et al. “Scientific reports, 6.1 (2016): 1-13).

The similarities in the basic biology of various TLR and other innate immune receptors suggest that a biomarker for response to a TLR9 agonist may have utility in predicting response not only to other TLR9 agonists, but also to other TLR agonists more generally (including especially TLR3, TLR4, TLR7, and TLR8), and to other innate immune activators known to those expert in the art, including for example cGAS/STING and RIG-I agonists, or activators of LGP2, MDA5, PKR, and/or AIM2.

The mechanisms regulating TLR9 translocation from the ER to the Golgi network and other intracellular compartments are likely to be important in regulating immune responses in vitro and in vivo, but until recently, no biomarkers associated with these processes had been identified in tumor biopsies of cancer patients.

Additionally, the present disclosure provides other transcriptional signatures that, either alone or in combination with the Golgi signature, can be used to determine whether a patient will benefit from, for example, a TLR9-based cancer therapy. For example, high expression of the transcription factor of ELF2 is associated with response to vidutolimod across baseline hot and cold tumors, and the expression of ELF2 can be combined with the enrichment of the Golgi signature to achieve greater predictive value for response to vidutolimod than either biomarker alone in a population of cancer patients.

### Other Biomarkers

In some embodiments, identifying a subject that is likely to benefit from a cancer immunotherapy comprising an innate immune activator comprises detection of the vidutolimod core signature gene transcription products in combination with one or more other prognostic factors or biomarkers. Transcriptional signatures represent one example of prognostic factors. These other prognostic factors or biomarkers can be used in combination with one or more of the Golgi signatures to provide more accurate prediction of the probability of response to vidutolimod alone or in combination with other immunotherapies such as PD-1 blockade.

Other transcriptional signatures or biomarkers that can be used to determine that a patient is likely to benefit from an innate immune activator such as a TLR9-based cancer therapy that are included in the invention are for example, low baseline frequency of macrophage/monocytes (using any of several published transcriptional signatures), low baseline T_{reg} signature (specifically, T_cell_regulatory_(T_{regs})_CIBERSORT), and moderate/low B cell-derived GABA signature. Individual genes that can comprise a part of a biomarker for this purpose include TLR8, TGFB2, TNFRSF1A, TNFRSF1B, C1QC, and/or PD-L1, especially in baseline “hot” tumors or TLR9 and/or TNFA, especially in baseline “cold” tumors, where relatively low levels of expression of one or more of these genes is used to identify patients more likely to respond to treatment with an innate immune activator.

Methods of Treating Patients that Present Transcriptional or Other Biomarkers

Vidutolimod (CMP-001) is a first-in-class CpG-A TLR9 agonist in a virus-like particle that activates plasmacytoid dendritic cells (pDC), bridging innate and adaptive immunity. Intratumoral (IT) injection of vidutolimod can function as an in situ immunization, reprograming the tumor microenvironment to promote the generation of anti-tumor CD8⁺ T cells with systemic responses to neoantigens. Vidutolimod has shown evidence of anti-tumor activity alone and/or in combination with PD-1 blockade in PD-1 refractory and/or neoadjuvant melanoma (NCT02680184 and NCT03618641), NSCLC (NCT03438318), and HNSCC (NCT02554812), has also been evaluated in MSS CRC (NCT03507699).

In some embodiments, CpG ODN of the disclosure are characterized, at least in part, by their propensity to induce high amounts of type I IFN. Exemplary CpG ODNs are CpG-A class molecules as described in PCT/US2015/067269 which is incorporated by reference herein in its entirety. In various embodiments of the present disclosure, virus-like particles (VLP) are used to formulate one or more CpG ODN. PCT/US2015/067269 described the use of VLPs with A-class CpGs, including CMP-001, which is specifically contemplated herein.

Virus capsids provide a protective shell of various sizes to most natural viruses (Mannige, R. V., and Brooks, C. L., PLOSONE, 5 (3): c9423 (2010); Perlmutter, J. D., and Hagan, M. F., J. Mol. Biol., 427 (15): 2451-2467 (2015). Structural information is also available for QB, a ssRNA phage (Gorzlenik, K. V., et al., PNAS, 113 (41): 11519-11524 (2016). Various groups have looked at the stabilization of VLPs in formulations (Lang, r., and Winter, G., Drug Devt. And Industr. Pharm., 35:83-97 (2009); Mertens, B. S., and Velev, O.D., Soft Matter, 11 (44): 8621-8631 (2015); and Shi, L., et al., J. Pharm. Sci., 94 (7): 1538-1551 (2005)).

By the term “therapeutically effective amount,” as used herein, is meant an amount that when administered to a mammal, preferably a human, mediates a detectable therapeutic response compared to the response detected in the absence of the compound. A therapeutic response, such as, but not limited to, inhibition of and/or decreased tumor growth (including tumor size stasis), tumor size, metastasis, and the like, can be readily assessed by a plethora of art-recognized methods, including, e.g., such methods as disclosed herein.

A “therapeutically effective amount” is intended to qualify the amount of an agent required to detectably reduce to some extent one or more of the symptoms of a neoplastic disorder, including, but not limited to: 1) reduction in the number of cancer cells; 2) reduction in tumor size; 3) inhibition (i.e., slowing to some extent, preferably stopping) of cancer cell infiltration into peripheral organs; 4) inhibition (i.e., slowing to some extent, preferably stopping) of tumor metastasis; 5) inhibition, to some extent, of tumor growth; 6) relieving or reducing to some extent one or more of the symptoms associated with the disorder; and/or 7) relieving or reducing the side effects associated with the administration of anticancer agents.

Certain preferred CpG ODN induce high or large amounts of type I IFN. Assays for measuring type I IFN are well known in the art and include in vitro enzyme-linked immunosorbent assay (ELISA) and cell-based assays, such as are described herein. Without meaning to be limiting, large or high amounts of type I IFN can refer to greater than or equal to about 1000 μg/mL IFN-α as measured according to such in vitro assays. In certain embodiments, large or high amounts of type I IFN can refer to greater than or equal to about 2000 μg/mL IFN-α as measured according to such in vitro assays. In certain embodiments, large or high amounts of type I IFN can refer to greater than or equal to about 3000 μg/mL IFN-α as measured according to such in vitro assays. In certain embodiments, large or high amounts of type I IFN can refer to greater than or equal to about 4000 μg/mL IFN-α as measured according to such in vitro assays. In certain embodiments, large or high amounts of type I IFN can refer to greater than or equal to about 5,000 μg/mL IFN-α as measured according to such in vitro assays.

Except when noted, the terms “patient” or “subject” are used interchangeably and refer to mammals such as human patients and non-human primates, as well as veterinary subjects such as rabbits, rats, and mice, and other animals. Preferably, “patient” or “subject” refers to a human. In certain embodiments, a subject is an adult human. In certain embodiments, a subject is a child. In certain embodiments, a subject is less than about 18 years of age. In certain embodiments, a subject is less than about 12 years of age.

As used herein, to “treat” means reducing the frequency with which symptoms of a disease (i.e., tumor growth and/or metastasis, or other effect mediated by the numbers and/or activity of immune cells, and the like) are experienced by a patient. Treatment may be prophylactic (to prevent or delay the onset of the disease, or to prevent the manifestation of clinical or subclinical symptoms thereof) or therapeutic suppression or alleviation of symptoms after the manifestation of the disease. The term “treat” includes the administration of the compounds or agents of the present disclosure to (i) prevent or delay the onset of the symptoms, complications, or biochemical indicia of, (ii) alleviate the symptoms of, and/or (iii) inhibit or arrest the further development of, the disease, condition, or disorder.

CpG oligonucleotides include at least one unmethylated CpG dinucleotide. An oligonucleotide containing at least one unmethylated CpG dinucleotide is an oligonucleotide molecule which contains a cytosine-guanine dinucleotide sequence (i.e., “CpG DNA” or DNA containing a 5′ cytosine linked by a phosphate bond to a 3′ guanine) and activates the immune system. The entire CpG oligonucleotide can be unmethylated or portions may be unmethylated, but at least the C of the 5′ CG 3′ must be unmethylated.

CpG ODN are generally about 8-100 nucleotides long. In certain embodiments, CpG ODN are about 8-50 nucleotides long, about 8-40 nucleotides long, about 8-30 nucleotides long, about 8-24 nucleotides long, about 8-20 nucleotides long, or about 8-16 nucleotides long.

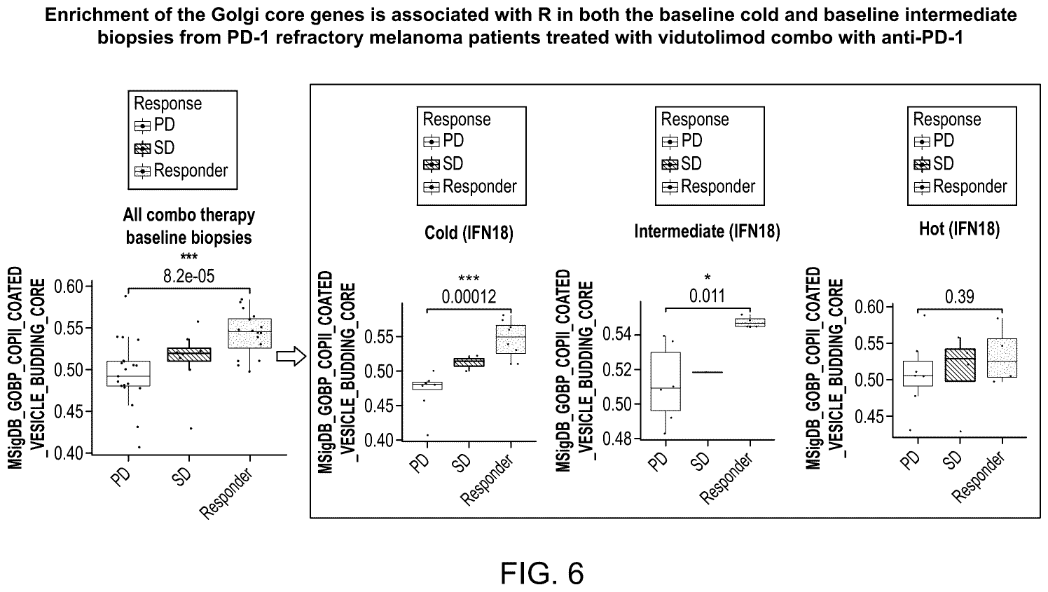

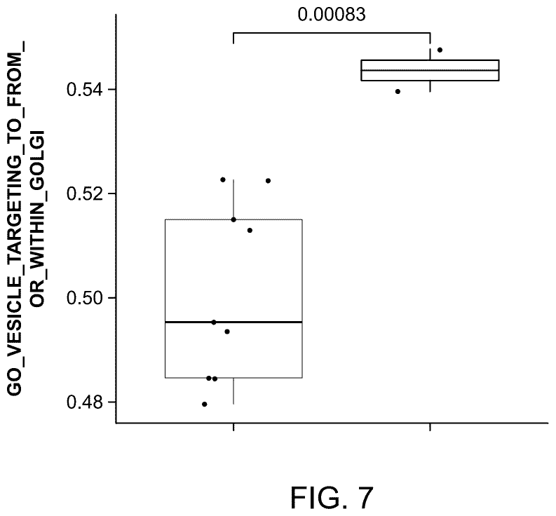

As described herein, in some embodiments CpG ODN of the disclosure are characterized, at least in part, by their propensity to induce high amounts of type I IFN. Exemplary CpG ODNs, for example, are CpG-A class molecules as described in PCT/US2015/067269 which is incorporated by reference herein in its entirety. Specifically, completely PO ODN G10 (SEQ ID NO: 82 and labeled as “CYT003” in FIGS. 6 and 7 of PCT/US2015/067269) is contemplated herein:

(SEQ ID NO: 1)
GGGGGGGGGGGACGATCGTCGGGGGGGGGG

As described herein, the methods as described herein may further comprise, in various embodiments, radiotherapy, chemotherapy, surgery, and/or administration of cyclophosphamide, a small molecule inhibitor, and/or a checkpoint inhibitor including, but not limited to an inhibitor of CTLA-4, 4-1BB (CD137), 4-1BBL (CD137L), PDL1 (PD-L1), PDL2, PD1, B7-H3, B7-H4, BTLA, HVEM, TIM3, GAL9, LAG3, TIM3, B7H3, B7H4, VISTA, KIR, BTLA, SIGLEC9, and 2B4, and checkpoint inhibitors such as, but not limited to, pembrolizumab, avelumab, atezolizumab, cetrelimab, dostarlimab, cemiplimab, spartalizumab, camrelizumab, durvalumab, or nivolumab.

The present disclosure relates to combination tumor immunotherapy comprising locally administering, in one embodiment, CpG ODN into or in proximity to a cancerous tumor, and systemically administering a checkpoint inhibitor, such as an anti-PD-1 antibody, an anti-PD-L1 antibody, or an anti-CTLA-4 antibody, to treat cancer.

Combination of high IFN-inducing CpG ODN and anti-PD-1, anti-PD-L1, or anti-CTLA-4 is useful for treatment of primary and secondary (i.e., metastatic) cancers. More specifically, among many potential treatment options, CpG ODN and anti-checkpoint combination therapy can be used to treat cancer. In certain embodiments, the cancer to be treated is or includes a cancerous tumor. A “cancerous tumor” as used herein refers to an abnormal swelling or macroscopic collection of cells comprising abnormal cells characterized by their growth or proliferation without regulation by normal external signals. In certain embodiments, a cancerous tumor is a carcinoma, sarcoma, or adenocarcinoma; these are sometimes referred to as solid tumors. In certain embodiments, a cancerous tumor excludes hematologic malignancies. In certain embodiments, a cancerous tumor includes certain hematologic malignancies, e.g., lymphomas.

Representative cancers treatable by the methods of the present disclosure (e.g., methods of treating cancer by administration of one or more CpG-ODN alone or in combination with one or more CPI) specifically include, without limitation, cancers of skin, head and neck, esophagus, stomach, liver, colon, rectum, pancreas, lung, breast, cervix, ovary, kidney, bladder, prostate, thyroid, brain, muscle, and bone. Also specifically included among cancers treatable by the methods of the invention are melanoma, renal cell carcinoma, and non-small cell lung cancer (NSCLC). Also specifically included among cancers treatable by the methods of the invention are lymphoma, cancer of the bone marrow, carcinoid tumor, and neuroblastoma.

While in some embodiments the foregoing cancers are preferred, the present disclosure relates to treatment of a wide variety of malignant cell proliferative disorders, including, but not limited to Kaposi's sarcoma, synovial sarcoma, mesothelioma, hepatobiliary (hepatic and biliary duct), a primary or secondary brain tumor, lung cancer (NSCLC and SCLC), bone cancer, skin cancer, cancer of the head or neck, cutaneous or intraocular melanoma, cancer of the anal region, stomach (gastric) cancer, gastrointestinal (gastric, colorectal, and duodenal) cancer, colon cancers, uterine cancer, carcinoma of the fallopian tubes, carcinoma of the endometrium, carcinoma of the cervix, carcinoma of the vagina, carcinoma of the vulva, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, prostate cancer, cancer of the penis, testicular cancer, cancer of the bladder, cancer of the kidney or ureter, carcinoma of the renal pelvis, pancreatic cancers, neoplasms of the central nervous system (CNS) including primary or secondary CNS tumor, spinal axis tumors, brain stem glioma, glioblastoma, meningioma, myoblastoma, astrocytoma, pituitary adenoma, adrenocortical cancer, gall bladder cancer, cholangiocarcinoma, fibrosarcoma, neuroblastoma, and retinoblastoma; as well as, in some embodiments, non-Hodgkin's lymphoma (NHL, including indolent and aggressive), Hodgkin's lymphoma, cutaneous T-cell lymphoma, lymphocytic lymphomas, primary CNS lymphoma, chronic or acute myeloid leukemia, chronic or acute lymphocytic leukemia, erythroblastoma, and multiple myeloma; or a combination of two or more of the foregoing cancers.

“Tumor” as it applies to a subject diagnosed with, or suspected of having, a cancer refers to a malignant or potentially malignant neoplasm or tissue mass of any size, and includes primary tumors and secondary neoplasms. A solid tumor is an abnormal growth or mass of tissue that usually does not contain cysts or liquid areas. Different types of solid tumors are named for the type of cells that form them. Examples of solid tumors are sarcomas, carcinomas, and lymphomas. Leukemias (cancers of the blood) generally do not form solid tumors (National Cancer Institute, Dictionary of Cancer Terms).

“Tumor burden” also referred to as “tumor load”, refers to the total amount of tumor material distributed throughout the body. Tumor burden refers to the total number of cancer cells or the total size of tumor(s), throughout the body, including lymph nodes and bone narrow. Tumor burden can be determined by a variety of methods known in the art, such as, e.g. by measuring the dimensions of tumor(s) upon removal from the subject, e.g., using calipers, or while in the body using imaging techniques, e.g., ultrasound, bone scan, computed tomography (CT) or magnetic resonance imaging (MRI) scans.

The term “tumor size” refers to the total size of the tumor which can be measured as the length and width of a tumor. Tumor size may be determined by a variety of methods known in the art, such as, e.g. by measuring the dimensions of tumor(s) upon removal from the subject, e.g., using calipers, or while in the body using imaging techniques, e.g., bone scan, ultrasound, CT or MRI scans.

The cancers to be treated may be refractory cancers, for example refractory to PD-1. A refractory cancer as used herein is a cancer that is resistant to the ordinary standard of care prescribed. These cancers may appear initially responsive to a treatment (and then recur), or they may be completely non-responsive to the treatment. The ordinary standard of care will vary depending upon the cancer type, and the degree of progression in the subject. It may be a chemotherapy, an immunotherapy, surgery, radiation, or a combination thereof. Those of ordinary skill in the art are aware of such standards of care. Subjects being treated according to the invention for a refractory cancer therefore may have already been exposed to another treatment for their cancer. Alternatively, if the cancer is likely to be refractory (e.g., given an analysis of the cancer cells or history of the subject), then the subject may not have already been exposed to another treatment.

In certain embodiments, refractory cancers include cancers which are refractory to treatment with a checkpoint inhibitor. Cancers of this type are sometimes referred to as “cold”. Methods of the instant invention can be used to treat such “cold” cancers or tumors to convert them into “hot” ones, i.e., cancers or tumors which respond to treatment, including treatment with a checkpoint inhibitor, even the same checkpoint inhibitor.

Examples of refractory cancers include but are not limited to melanomas, renal cell carcinomas, colon cancer, liver (hepatic) cancers, pancreatic cancer, non-Hodgkin's lymphoma, other lymphomas, lung cancer, prostate cancer, breast cancer, and leukemias.

In accordance with the methods of the present disclosure, a cancer therapy described herein is, in various embodiments, administered locally to the cancerous tumor, i.e., by intratumoral or peritumoral administration. Alternatively or in addition, in certain embodiments a cancer therapy comprising a TLR9 agonist, such as CMP-001, is administered locally to the cancerous tumor by, for example, intraperitoneal injection or infusion or intravesicular instillation. As will be appreciated by the ordinarily skilled artisan, the cancer therapy comprising a TLR9 agonist, such as CMP-001, dosing described herein includes, in a preferred embodiment, dosing amounts and regimens in the context of administration via a VLP, such as Qβ and CMP-001, as described herein. In this way, the VLP comprises bacteriophage Qβ coat protein in an amount of about 1 mg to about 100 mg. In other embodiments, the VLP comprises bacteriophage Qβ coat protein in an amount of about 1, 2, 3, 4, 5, 6, 7, 8, 9, 10, 15, 25, 50, or 100 mg. In other embodiments, 1.56 mg, about 3.125 mg, about 6.25 mg, about 12.5 mg, about 25 mg, about 50 mg, or about 100 mg are contemplated.

In some embodiments, about 80 oligonucleotides, e.g., G10 or other CpG ODN described herein, or approximately 760 kDa MW CpG ODN aggregate, are packaged in a VLP. In some embodiments, 1, 5, 10, 50, 80, 100, 120, 150, or 200 or more oligos are packaged.

The immune effects of XRT given prior to cancer therapy comprising a TLR9 agonist, such as CMP-001, administration will disrupt the inhibitory mechanisms that normally limit the efficacy of the CpG-induced response, increasing the potential for clinical response. In addition, the production of IFN-α in the tumor has been associated with and is required for an improved response to XRT (Burnette et al, Cancer Res. 2011 71: 2488-2496), providing further evidence for benefit from the use of intratumoral high IFN CpG following XRT. The timing of administration of XRT and TLR9 agonist can vary. In some embodiments the XRT is given at least one month prior to the TLR9 agonist, or at least 2 weeks, or at least 1 week prior to the TLR9 agonist. In other embodiments the XRT is administered to new lesions that occur during TLR9 agonist therapy, and the TLR9 agonist is injected into the newly irradiated lesion before, after, or during the XRT.

Methods of intratumoral or peritumoral delivery of cancer therapy comprising a TLR9 agonist, such as CMP-001, include not only direct injection, but also can include topical delivery intraperitoneal delivery for abdominal tumors such as ovarian, pancreatic, colon, or gastric), intraocular for eye malignancies, oral for gastric and intestinal cancer, and intravesicular administration for bladder cancer. Also contemplated for intratumoral administration of cancer therapy comprising a TLR9 agonist, such as CMP-001, is systemic delivery using tumor delivery vehicles such as tumor-targeted aptamers, antibody conjugates, nanoparticles, ISCOMS, VLP, multilaminar vesicles, pH-sensitive peptides, and cationic peptides.

In certain embodiments, subject doses of CpG ODN for intratumoral and peritumoral delivery typically range from about 10 μg to about 100 mg per administration, which depending on the application could be given daily, weekly, or monthly and any other amount of time therebetween. In certain embodiments, subject doses of cancer therapy comprising a TLR9 agonist, such as CMP-001, for intratumoral and peritumoral delivery typically range from about 100 μg to about 100 mg per administration, which depending on the application could be given daily, weekly, or monthly and any other amount of time there between. In certain embodiments, subject doses of cancer therapy comprising a TLR9 agonist, such as CMP-001, for intratumoral and peritumoral delivery typically range from about 1 mg to about 100 mg per administration, which depending on the application could be given daily, weekly, or monthly and any other amount of time therebetween. In certain embodiments, subject doses of cancer therapy comprising a TLR9 agonist, such as CMP-001, for intratumoral and peritumoral delivery typically range from about 10 mg to about 100 mg per administration, which depending on the application could be given daily, weekly, or monthly and any other amount of time therebetween.

Certain commercially available anti-PD-1 antibodies are currently approved in the United States for intravenous infusion dosing at 2 mg/kg body weight once every three weeks. Other commercially available anti-PD-1 antibodies are currently approved in the United States for intravenous infusion dosing at 3 mg/kg body weight once every two weeks. Commercially available anti-CTLA-4 antibodies are currently approved in the United States for intravenous infusion dosing at 3 mg/kg body weight once every three weeks.

In accordance with the methods of the present disclosure, in certain embodiments, CPI antibody is administered, at least in part, systemically, e.g., intravenously.

Unless otherwise defined herein, scientific and technical terms used in connection with the present disclosure shall have the meanings that are commonly understood by those of ordinary skill in the art. Further, unless otherwise required by context, singular terms shall include pluralities and plural terms shall include the singular. Generally, nomenclatures used in connection with, and techniques of, cell and tissue culture, molecular biology, immunology, microbiology, genetics and protein and nucleic acid chemistry and hybridization described herein are those well-known and commonly used in the art.

The methods and techniques of the present disclosure are generally performed according to methods well known in the art and as described in various general and more specific references that are cited and discussed throughout the present specification unless otherwise indicated. Such references include, e.g., Sambrook and Russell, Molecular Cloning, A Laboratory Approach, Cold Spring Harbor Press, Cold Spring Harbor, N.Y. (2001), Ausubel et al., Current Protocols in Molecular Biology, John Wiley & Sons, NY (2002), and Harlow and Lane Antibodies: A Laboratory Manual, Cold Spring Harbor Laboratory Press, Cold Spring Harbor, N.Y. (1990), which are incorporated herein by reference. Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

As used herein, each of the following terms has the meaning associated with it in this section.

The articles “a” and “an” are used herein to refer to one or to more than one (i.e., to at least one) of the grammatical object of the article. As used herein, the twenty conventional amino acids and their abbreviations follow conventional usage. See Immunology—A Synthesis (2nd Edition, E. S. Golub and D. R. Gren, Eds., Sinauer Associates, Sunderland, Mass. (1991)), which is incorporated herein by reference. Conventional notation is used herein to portray polypeptide sequences: the left-hand end of a polypeptide sequence is the amino-terminus; the right-hand end of a polypeptide sequence is the carboxyl-terminus. A “conservative amino acid substitution” is one in which an amino acid residue is substituted by another amino acid residue having a side chain R group with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution. Means for making this adjustment are well-known to those of skill in the art. See, e.g., Pearson, Methods Mol. Biol. 243:307-31 (1994).

Having now described the present disclosure in detail, the same will be more clearly understood by reference to the following examples, which are included herewith for purposes of illustration only and are not intended to be limiting of the invention.

### EXAMPLES

PCT/US2015/067269 discloses several A-class CpG ODN, each of which are contemplated and incorporated by reference herein. As used herein, CMP-001 refers to an A-class CpG-ODN, referred to as G10 and SEQ ID NO: 1, that is formulated in a VLP. CMP-001 has also been known as CYT003 and as QbG10, under which names it has previously been studied in mice and humans for non-oncology indications, for example in Klimek, L., et al., Clinical & Experimental Allergy, 41.9 (2011): 1305-1312, and Casale, T. B., et al., Allergy, 70.9 (2015): 1160-1168.

### Example 1

A. Vesicle-Golgi Transporting Signatures (“Golgi Signature”) are Associated with Clinical Response to Vidutolimod (CMP-001) in PD-1 Refractory Melanoma

The design and outcomes measures for the clinical trial NCT02680184 of intratumoral vidutolimod in PD-1 refractory melanoma, including both monotherapy and combination with pembrolizumab treatment groups have been described previously (Kirkwood, John, et al. “950 Final analysis: phase 1b study investigating intratumoral injection of toll-like receptor 9 agonist vidutolimod±pembrolizumab in patients with PD-1 blockade-refractory melanoma,” SITC 2021)). Vidutolimod therapy was either undiluted (“PS20 A” in Kirkwood et al., SITC 2021) or diluted in 0.9% saline (“PS20 B” in Kirkwood et al., SITC 2021). RNA-seq data from archival or screening baseline/pre-treatment tumor biopsies of patients in this clinical trial) were processed and normalized using standard methods. This data set included in the analyses of this trial comprised 98 baseline biopsies (Table A) from patients subsequently receiving vidutolimod monotherapy or combination therapy with pembrolizumab by subsequent responder status following this treatment as assessed using RECIST v1.1 (CR=complete response; PR=partial response; SD=stable disease; PD=progressive disease). Responder (R) indicates CR or PR, while nonresponders (NR) indicates SD or PD.

TABLE A
CMP-001-001 baseline samples with RNAseq

| | | Combo | | Mono | |
|---|---|---|---|---|---|
| | | | # of | | # of |
| | # of all | # of all | undiluted | # of all | undiluted |
| RECIST | samples | combo | combo | mono | mono |
| CR | 5 | 5 | 3 | 0 | 0 |
| PR | 15 | 11 | 7 | 4 | 1 |
| SD | 30 | 22 | 8 | 8 | 7 |
| PD | 48 | 40 | 21 | 8 | 5 |
| NA | 7 | 6 | 2 | 1 | 1 |
| (not included in | | | | | |
| the analysis) | | | | | |

Responder: CR + PR

In initial efforts to identify a gene or gene expression signature associated with response, a portion of the full RNA Seq dataset was analyzed using standard bioinformatic approaches to identify baseline predictors for response. Although many differences were found in gene expression between responders and non-responders, including the identification of candidate response signatures, none of the candidate signatures or gene expression differences were validated in the full dataset to significantly distinguish responders from non-responders.

These analyses showed that gene signatures thought to reflect “hot” or “inflamed” tumors, which had been previously reported to be associated with response to PD-1 blockade, such as the IFN18 signature of Ayers et al. (WO/2016/094377), or TIDE, or the biopsy IHC combined pathologic score (CPS) for PD-L1, or tumor mutations were not associated with vidutolimod response in the dataset of PD-1 refractory melanoma (Luke, Jason John, et al. “Abstract CT032: CMP-001 demonstrates improved response in noninflamed anti-PD-1 refractory melanoma and response is associated with serum CXCL10.” (SITC 2021): CT032-CT032). This result was expected, based on the unique mechanism of vidutolimod. As described herein, in one embodiment a tumor is determined to be “not hot” using one or more of the aforementioned and techniques described herein, including TIDE, Immunoscore, the IFN18 signature of Ayers et al. (WO/2016/094377), and/or the biopsy IHC combined pathologic score (CPS) for PD-L1.

These studies with published markers for tumor “inflammation” such as TIDE and the IFN18 signature revealed a high degree of heterogeneity within the examined patient population, with most of the patients having “cold” or non-inflamed tumors, others being “hot,” or inflamed, and the remainder being intermediate at baseline. Interestingly, there have been no reports of any signature in baseline “cold” tumors that is associated with response to an immunotherapeutic agent. To identify potential signatures that could be unique to the baseline “cold”, “hot”, or “intermediate” tumors, published tools including TIDE, IFN18, and CPS were used to divide the set of baseline biopsies described above into three subsets.

Initially selecting just the baseline “cold” and “hot” biopsy datasets, enrichment scores were calculated for all of the signatures from the Molecular Signatures Database (MSigDB; N=18,494) and for signatures developed by Checkmate (N=555) using ssGSEA (single-sample GSEA) in each sample and identified the dys-regulated signatures between Responder (CR and PR) vs. Non-responders (PD or SD) using Limma. The results of this analysis using IFN18 to select the baseline cold biopsies are shown in the Table B below, with highlighting of the signatures relating to vesicle trafficking and Golgi transport or function.

TABLE B

| | logFC_CRPRvsPD | P.Value_CRPRvsP [symbol] |
|---|---|---|
| REACTOME_DIGESTION_OF_DIETARY_CARBOHYDRATE | −0.078328705 | 0.000119789 |
| GO_POSITIVE_REGULATION_OF_GLYCOGEN_METABOLIC_PROCESS | −0.073686073 | 0.000305778 |
| REACTOME_ER_TO_GOLGI_ANTEROGRADE_TRANSPORT | 0.035302787 | 0.000630481 |
| REACTOME_COPI_MEDIATED_ANTEROGRADE_TRANSPORT | 0.035001466 | 0.000868625 |
| GO_COPII_COATED_VESICLE_BUDDING | 0.033795824 | 0.000894454 |
| HO_LIVER_CANCER_VASCULAR_INVASION | −0.062630092 | 0.000900287 |
| GO_GOLGI_VESICLE_BUDDING | 0.034238297 | 0.001023749 |
| REACTOME_COPII_MEDIATED_VESICLE_TRANSPORT | 0.032890572 | 0.001297778 |
| ZHOU_PANCREATIC_EXOCRINE_PROGENITOR | −0.09687645 | 0.001315872 |
| GO_VESICLE_TARGETING_TO_FROM_OR_WITHIN_GOLGI | 0.033133108 | 0.002130392 |
| GO_POSITIVE_REGULATION_OF_GLUCOSE_METABOLIC_PROCESS | −0.0517533 | 0.002259918 |
| GO_POSITIVE_REGULATION_OF_NEURON_PROJECTION_REGENERATION | 0.049475411 | 0.002268279 |
| GSE20152_SPHK1_KO_VS_WT_HTNFA_OVERXPRESS_ANKLE_UP | 0.028863434 | 0.002357438 |
| GO_ATRIAL_CARDIAC_MUSCLE_CELL_MEMBRANE_REPOLARIZATION | −0.05053516 | 0.002535502 |
| GO_VESICLE_BUDDING_FROM_MEMBRANE | 0.027791969 | 0.00327843 |
| GO_VESICLE_TARGETING | 0.030833041 | 0.003842458 |
| GO_NEUROPEPTIDE_Y_RECEPTOR_ACTIVITY | −0.077138285 | 0.004074298 |
| GSE11057_NAIVE_VS_EFF_MEMORY_CD4_TCELL_UP | −0.029777445 | 0.004252907 |
| GSE11864_CSF1_VS_CSF1_PAM3CYS_IN_MAC_DN | 0.023362178 | 0.005140106 |
| Checkmate_GSE93679_OXmtDNA_100_DN | −0.043132508 | 0.005261066 |
| MODULE_509 | 0.055324799 | 0.005504911 |
| GO_GOLGI_VESICLE_TRANSPORT | 0.024814253 | 0.005765166 |
| GO_INTRA_GOLGI_VESICLE_MEDIATED_TRANSPORT | 0.029806275 | 0.005769746 |
| GO_VOLTAGE_GATED_SODIUM_CHANNEL_COMPLEX | −0.087728412 | 0.006154394 |
| GO_VESICLE_CARGO_LOADING | 0.034520893 | 0.006372463 |
| GO_VESICLE_MEDIATED_TRANSPORT_TO_THE_PLASMA_MEMBRANE | 0.026884441 | 0.006861044 |
| GO_COPII_VESICLE_COAT | 0.042670254 | 0.006989679 |
| REACTOME_VOLTAGE_GATED_POTASSIUM_CHANNELS | −0.052644682 | 0.007169308 |
| GO_RETINAL_BINDING | −0.091008362 | 0.007244163 |
| GO_COPI_VESICLE_COAT | 0.050093399 | 0.007294348 |
| GSE5542_UNTREATED_VS_IFNA_AND_IFNG_TREATED_EPITHELIAL_CELLS_6H_UP | − 0.019491644 | 0.007575119 |
| GO_COPI_COATED_VESICLE_MEMBRANE | 0.043183716 | 0.008059267 |
| GO_POST_GOLGI_VESICLE_MEDIATED_TRANSPORT | 0.025541439 | 0.008396457 |
| GARGALOVIC_RESPONSE_TO_OXIDIZED_PHOSPHOLIPIDS_BLUE_UP | 0.037014874 | 0.008602205 |
| REACTOME_TRANSPORT_TO_THE_GOLGI_AND_SUBSEQUENT_MODIFICATION | 0.028450308 | 0.009315149 |
| GO_POTASSIUM_CHANNEL_ACTIVITY | −0.036340882 | 0.009547259 |
| GO_REGULATION_OF_VASCULAR_SMOOTH_MUSCLE_CELL_DIFFERENTIATION | −0.065043615 | 0.009563737 |
| GO_CARDIAC_VASCULAR_SMOOTH_MUSCLE_CELL_DIFFERENTIATION | −0.09707886 | 0.009593374 |
| GO_DELAYED_RECTIFIER_POTASSIUM_CHANNEL_ACTIVITY | −0.060884992 | 0.009693386 |
| GO_REGULATION_OF_NEUROMUSCULAR_JUNCTION_DEVELOPMENT | −0.06152006 | 0.010023364 |
| MARIADASON_REGULATED_BY_HISTONE_ACETYLATION_DN | 0.034735902 | 0.01006165 |
| xCell_Mesangial_cells_ENCODE_1 | −0.070261023 | 0.010245918 |
| GSE369_SOCS3_KO_VS_WT_LIVER_POST_IL6_INJECTION_UP | 0.021764736 | 0.010319952 |
| GO_COPII_COATED_VESICLE_CARGO_LOADING | 0.042040628 | 0.010334665 |
| GO_POSITIVE_REGULATION_OF_CELL_SIZE | 0.066660994 | 0.010625695 |
| GO_DENDRITE_MEMBRANE | −0.036636362 | 0.010632439 |
| GO_POTASSIUM_ION_TRANSMEMBRANE_TRANSPORTER_ACTIVITY | −0.032048585 | 0.010927375 |
| GO_POSTSYNAPTIC_ENDOSOME | 0.048004981 | 0.011148413 |
| MODULE_395 | 0.047322767 | 0.011189771 |

[symbol]

indicates data missing or illegible when filed

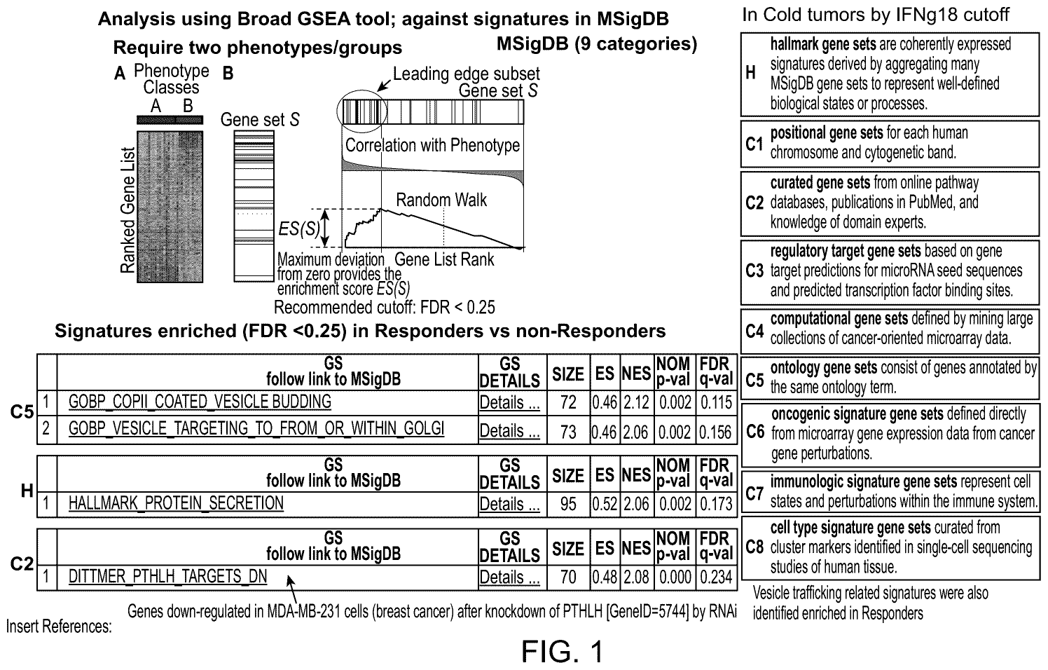

Among the top gene signatures most highly enriched in Responder vs. PD, were signatures that involved vesicle budding or targeting and Golgi transport (e.g. genes associated with COPII vesicle budding and/or vesicle trafficking to or from a Golgi body). Additional analyses were performed on data from “cold tumors” using GSEA (www.gsca-msigdb.org/gsea/index.jsp; two groups comparison) between Responder vs. PD against signatures in MSigDB (FIG. 1). Four gene signatures were identified that were enriched in Responders vs. PD (FDR<0.25, default cutoff), including COPII_COATED_VESICLE_BUDDING (73 genes) (FDR=0.115), VESICLE_TARGETING_TO_FROM_OR_WITHIN_GOLGI (74 genes) (FDR=0.156), HALLMARK_PROTEIN_SECRETION (96 genes) (FDR=0.173), and DITTMER_PTHLH_TARGETS_DN (70 genes) (FDR=0.234) (FIG. 1).

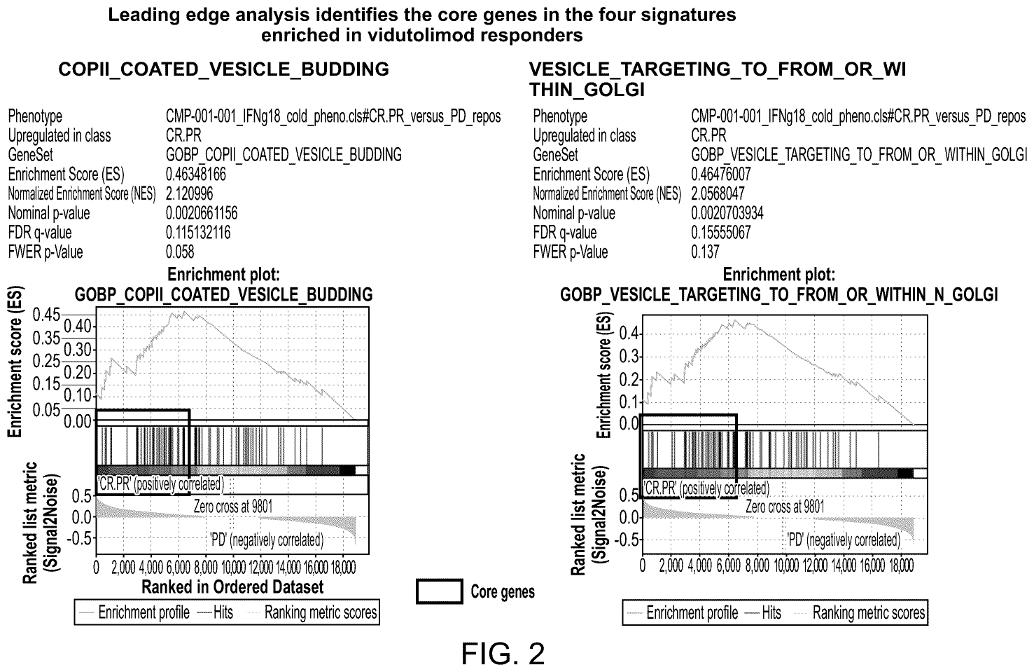

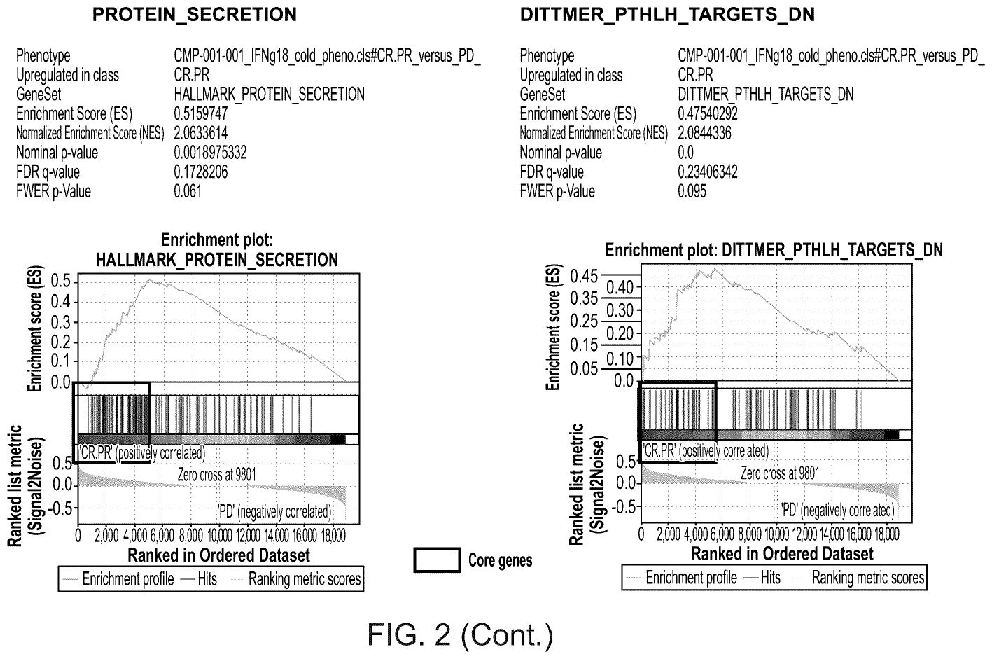

Leading edge analysis (a tool within GSEA; Subramanian, A., et al., PNAS, 2005, 102 (43): 15545-50) was performed in order to identify the core gene subset contributing the most to the enrichment signal for the four gene signatures (FIG. 2).

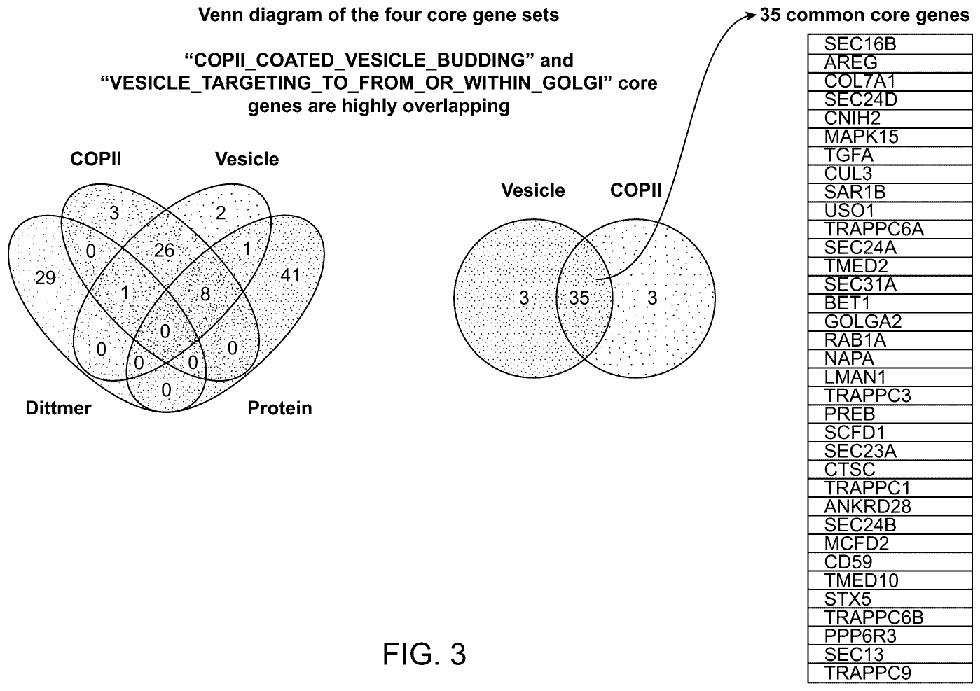

These core gene lists were then examined using a Venn diagram to detect shared common genes between the 4 core gene signatures. This Venn diagram (FIG. 3) showed minimal overlap between the Dittmer and Hallmark protein secretion gene lists and so these were dropped from further analysis.

By this means, the original number of genes were reduced down to define a novel highly overlapping “core gene set” from COPII_COATED_VESICLE_BUDDING and VESICLE_TARGETING_TO_FROM_OR_WITHIN_GOLGI containing 35 common genes (FIG. 3)—referred to herein as “common core”, with 3 additional unique genes for each signature making a total of 41 genes, including (the 6 unique genes are listed last): SEC16B, AREG, COL7A1, SEC24D, CNIH2, MAPK15, TGFA, CUL3, SAR1B, USO1, TRAPPC6A, SEC24A, TMED2, SEC31A, BET1, GOLGA2, RAB1A, NAPA, LMAN1, TRAPPC3, PREB, SCFD1, SEC23A, CTSC, TRAPPC1, ANKRD28, SEC24B, MCFD2, CD59, TMED10, STX5, TRAPPC6B, PPP6R3, SEC13, TRAPPC9, CEP19, ARFGAP3, GOSR1, SEC23B, MIA3, VAPA which together comprise a Golgi vidutolimod response signature, also referred to herein as the “Golgi signature” or the “vidutolimod response core signature.”

ssGSEA analyses were re-run for all >19,000 gene signatures, plus the newly identified four core signatures and “common core” in CMP-001-001 baseline samples. The top dys-regulated three signatures between Responder vs. PD for the baseline cold tumors of patients treated with vidutolimod monotherapy or in combination with pembrolizumab are: VESICLE_TARGETING_TO_FROM_OR_WITHIN_GOLGI_core (38 genes) (adj P=0.059), COPII_COATED_VESICLE_BUDDING_core (38 genes) (adj P=0.059), and common_core (35 genes) (adj P=0.085). The following additional evaluations were focused on these three core signatures.

### B. Enrichment of the Golgi Signature in Baseline Cold Responders to Both Combination Therapy (Vidutolimod+Pembrolizumab) and Vidutolimod Monotherapy.

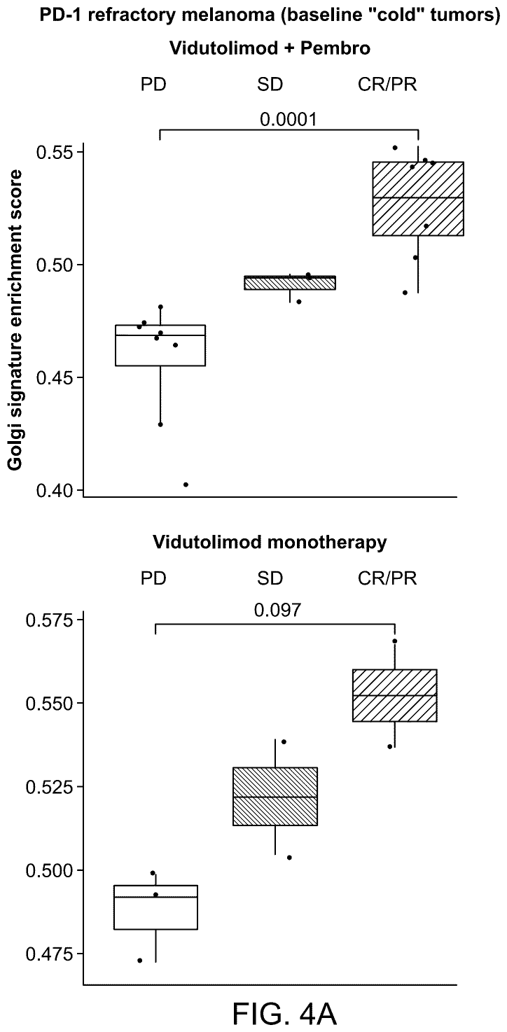

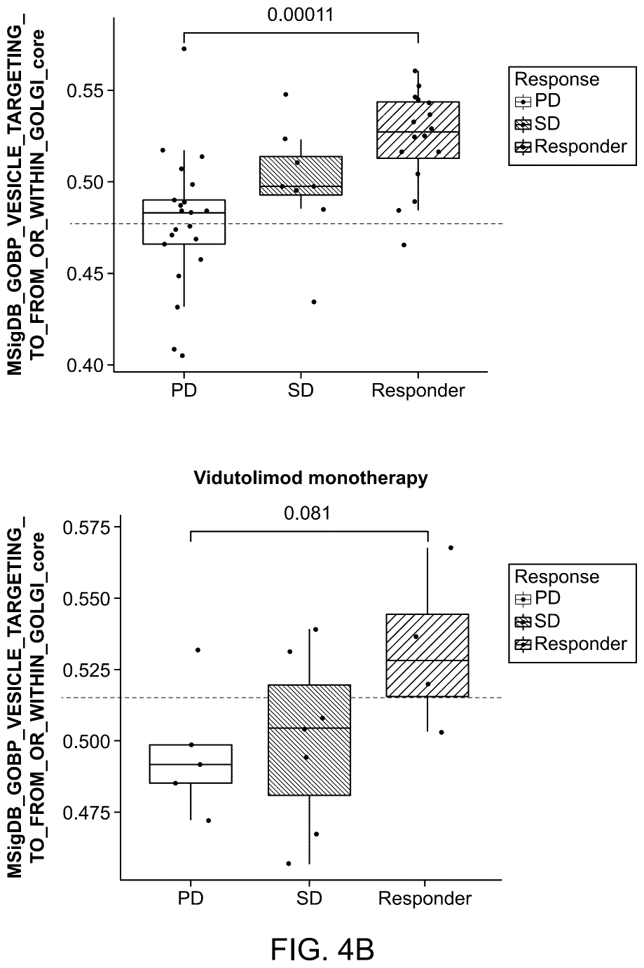

The original identification of the association of the Golgi signature expression with response was performed on the pooled data from all patients with baseline cold tumors who had been treated with undiluted vidutolimod, including patients receiving combination or monotherapy. Separate analyses of those subsets showed comparable association with response in both subsets, consistent with the hypothesis that the association reflects the biology of vidutolimod, rather than only the biology of the combination treatment (FIG. 4A). There was a similar strong trend to an association of the Golgi core signature with response when the total population of baseline biopsies was analyzed as a group (FIG. 4B).

### C. Lack of Enrichment of Common Core Golgi Signatures in the Baseline “Hot” Biopsies

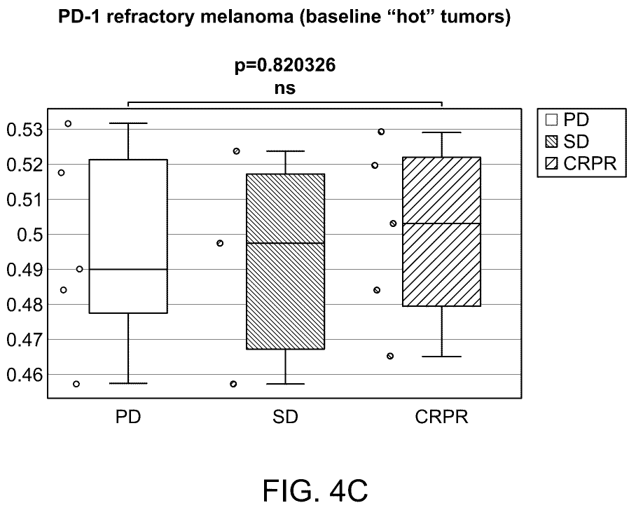

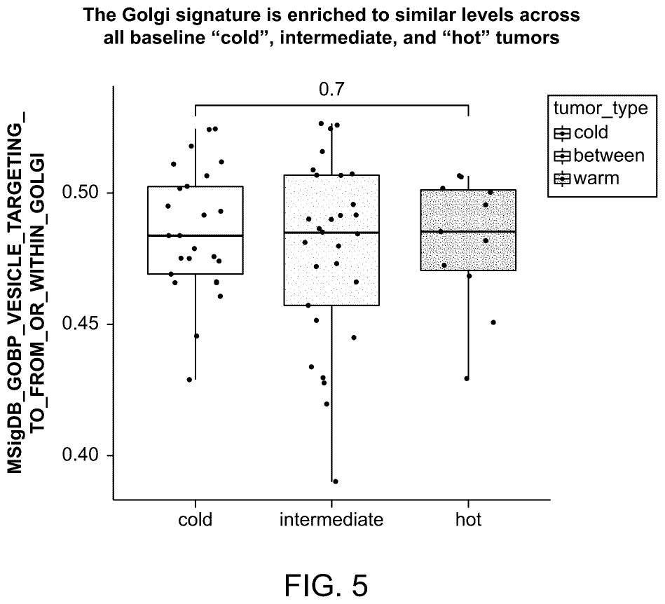

The biology of baseline “hot” and “cold” tumors is quite distinct, as illustrated for example by the fact that response to PD-1 blockade is much more frequent in baseline hot compared to cold tumors. Based on this well-established fact, we hypothesized that different baseline factors may predict response to vidutolimod in the baseline “cold” and “hot” tumors. Indeed the baseline “hot” tumors showed no association of Golgi signatures with response (FIG. 4C), consistent with the distinct biology of those tumors. The enrichment scores for the Golgi signatures were comparable across all baseline “cold” vs. “intermediate” vs. “hot” tumors, indicating that the signature is not a surrogate indicator for some inherent biologic difference between “cold” and “hot” tumors (FIG. 5).

### D. Validation of the Association of the Common Core and Golgi Vidutolimod Response Signature in Independent Datasets.

To determine whether the enrichment of Golgi signatures in the baseline “cold” undiluted tumors was unique to that dataset, the enrichment scores for the Golgi signatures were evaluated in independent subsets of the baseline biopsy RNA Seq data from patients with PD-(L)-1 refractory melanoma (clinical trial NCT02680184) or NSCLC (clinical trial NCT03438318) treated with vidutolimod±anti-PD-(L) 1. These data had not been used for the original identification of the Golgi signature, and therefore served as validation datasets to test the broader association of the signature with response.

In PD-1 refractory melanoma patients with baseline “intermediate” tumors treated with vidutolimod+pembrolizumab the Golgi signature is significantly enriched in responders vs. PD to a similar level as in the baseline “cold” tumors and the patients with SD are intermediate (FIG. 6; only one signature is shown; comparable significant associations were seen for the other signatures and the common core).

For independent validation of these findings in PD-1 refractory melanoma, the enrichment of Golgi signatures was also evaluated in baseline biopsies from patients with PD-1-refractory NSCLC treated with vidutolimod+atezolizumab. Two patients in that trial had tumor shrinkage and prolonged stable disease post treatment: these same two patients also had the highest enrichment of the Golgi signatures in their baseline tumors, which was well above the scores in the nonresponding patients (FIG. 7). These data provide independent confirmation for the validity of the Golgi signatures for predicting response to vidutolimod therapy from a baseline tumor biopsy.

### E. Scoring Methods for Defining the Enrichment Score for the Golgi Signature

In the above analyses, the scoring method is ssGSEA. While ssGSEA is more robust compared to expressionByMean/expressionByMedian, both expressionByMean and expresionByMedian scoring methods are contemplated and provided herein, in various embodiments.

### F. Methods of Grouping Cold/Hot Tumors

Different methods to group cold/hot tumors were evaluated: (1) IFNg_18 gene signature (Ayers, M., et al., J Clin Invest., 2017, 127 (8): 2930-2940), with low IFNg_18 score (<−0.5 in the current data and analysis) for “cold” tumor, high IFNg_18 score (>0.5) for “hot” tumor, and others for “intermediate” tumors. (2) TIDE score (https://pubmed.ncbi.nlm.nih.gov/30127393/): with high TIDE score (>1, predicted to not respond to CPI) for “cold” tumor, low TIDE score (<−1, predicted to respond to CPI) for “hot” tumor, and the others for “intermediate” tumors. (3) CPS score (PD-L1 IHC assay): Cold: CPS<1; intermediate: 1<CPS<10; Warm: CPS>10.

G. Lack of Association of the Golgi Vidutolimod Response Signatures with Response to CPI (Checkpoint Inhibitors)

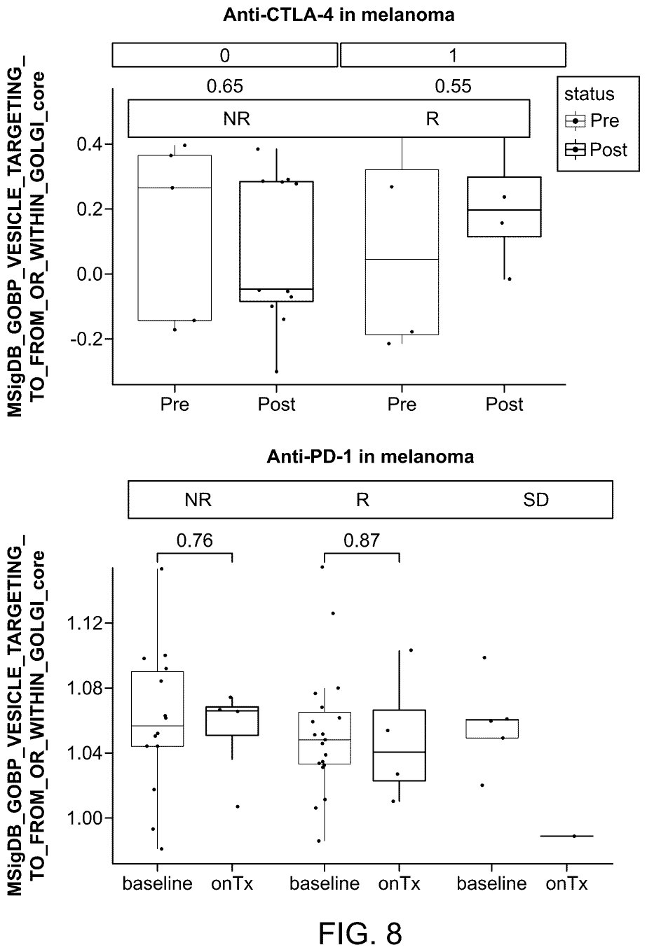

The enrichment of Golgi signatures was also evaluated in baseline samples from publicly available CPI treatment RNA Seq datasets. In one anti-CTLA-4 monotherapy (ipilimumab) study (Nathanson, T., et al., Cancer Immunol Res., 2017, 5 (1): 84-91), there is no association of Golgi signatures with response, nor there is an induction/reduction of Golgi signature post anti-CTLA4 treatment (FIG. 8). In a separate study with anti-PD-1 monotherapy (Gide, T. N., et al., Cancer Cell, 2019, 35 (2): 238-255) there is again no association of the Golgi core signature with clinical response, and no induction of the signature post-treatment, indicating that this signature is not a general marker predicting response to any cancer immunotherapy (FIG. 8).

H. Enrichment of Golgi Signatures in Plasma Cells, IFN-Secreting pDC, and Other Immune Cells

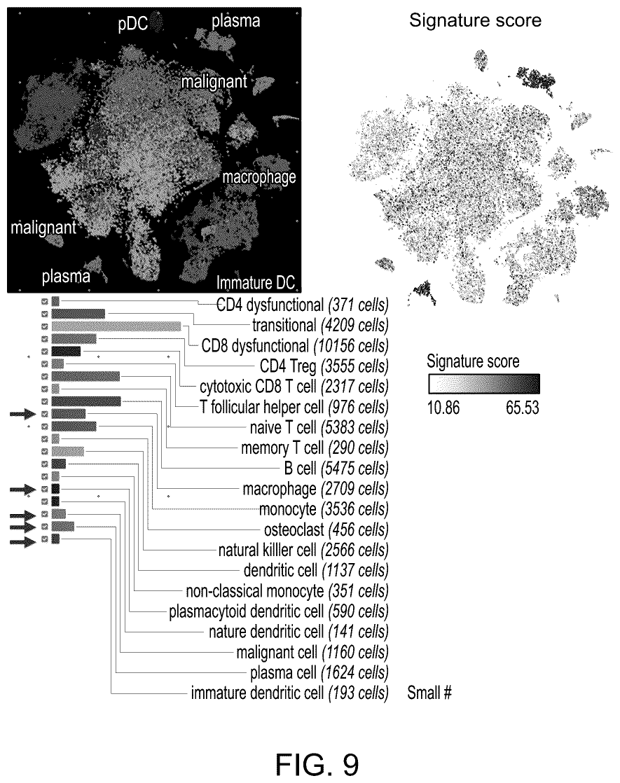

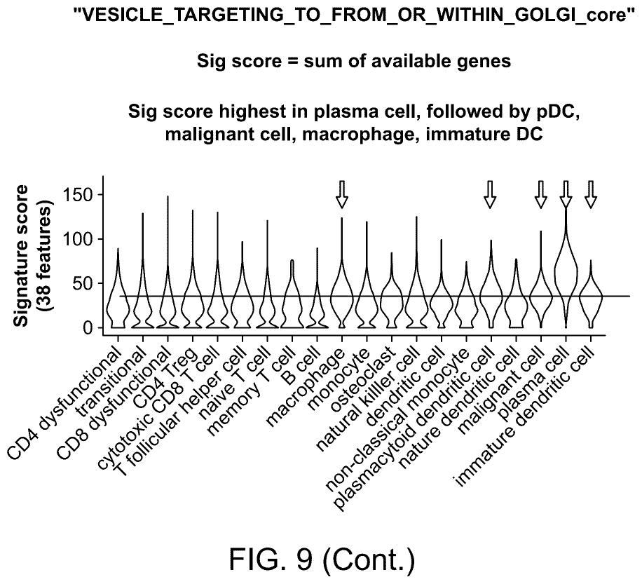

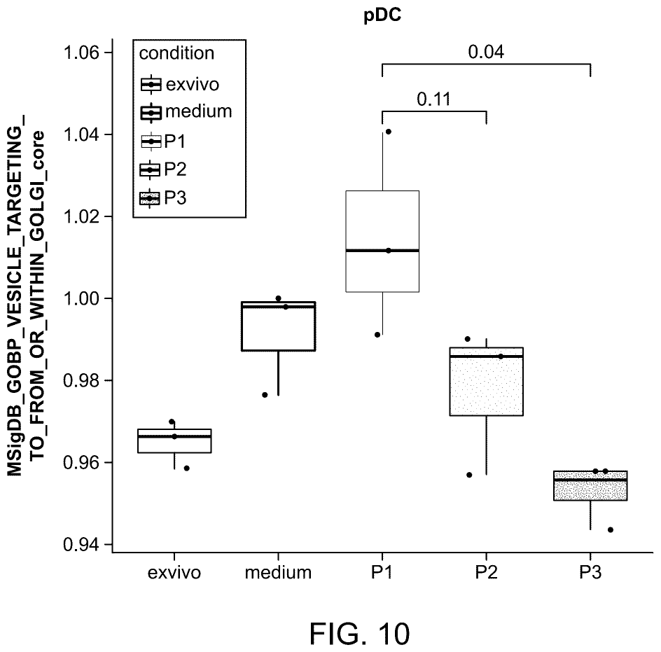

It was possible that the Golgi signature could reflect contributions from many different cells, and that no single cell might express the entire signature. In order to assess the enrichment of Golgi signatures in different individual immune cells, signatures were evaluated in proprietary and publicly available single cell RNA Seq data sets. In multiple public single cell datasets, the Golgi signature expression is highest in plasma cells, followed by pDC, macrophages, and several other immune cell populations (FIG. 9 is an example of typical data). In addition, in publicly available data for purified pDC activated in vitro into three distinct subsets (P1, P2, P3) the Golgi signatures are most highly expressed in the P1 subset which is the highest type I IFN producing pDC subset. (FIG. 10) These studies further relate the biology of the Golgi signature to the biology of TLR9 activation and the MOA of vidutolimod, which depends on the induction of type I IFN to drive anti-tumor immunity.

### I. Golgi Vidutolimod Response Signatures are Highly Expressed in Many Different Tumor Indications

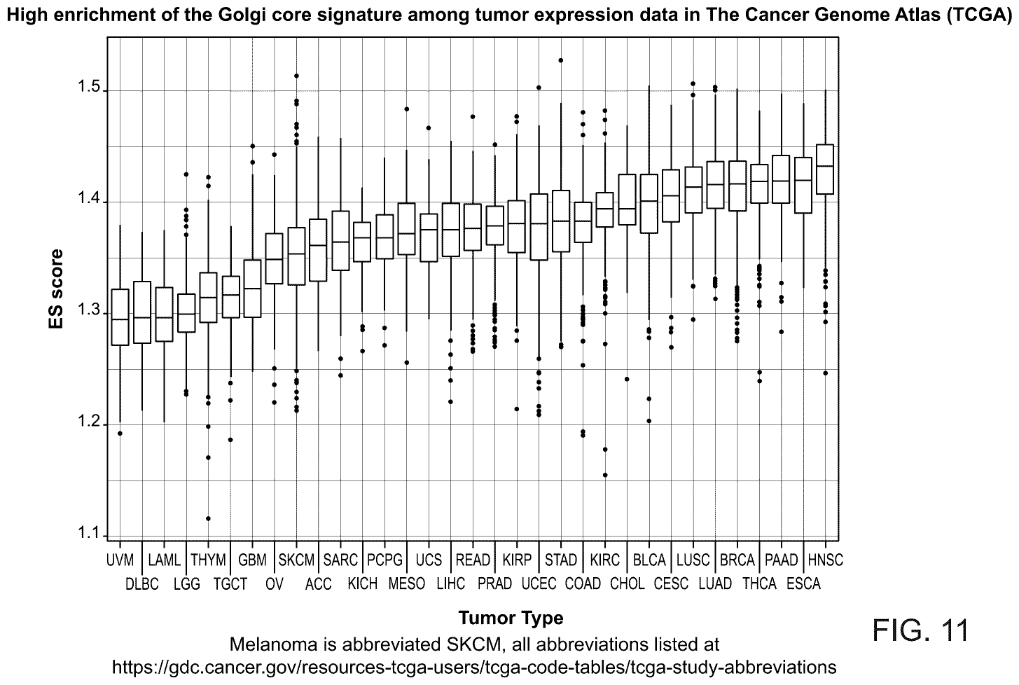

The enrichment of Golgi signatures was assessed in different tumor indications using the publicly available TCGA (FIG. 11). This analysis demonstrates that expression of the signature is not limited to melanoma (SKCM) or NSCLC, but is highly prevalent among most tumor types, indicating a great potential for the broad use of this signature alone or in combination with other biomarkers for selecting patients with cancer who have a high probability of responding to vidutolimod therapy, alone or in combination with other agents.

J. The Golgi Signatures May be Induced by Radiotherapy (XRT) in Combination with Standard of Care (SOC) Chemotherapy

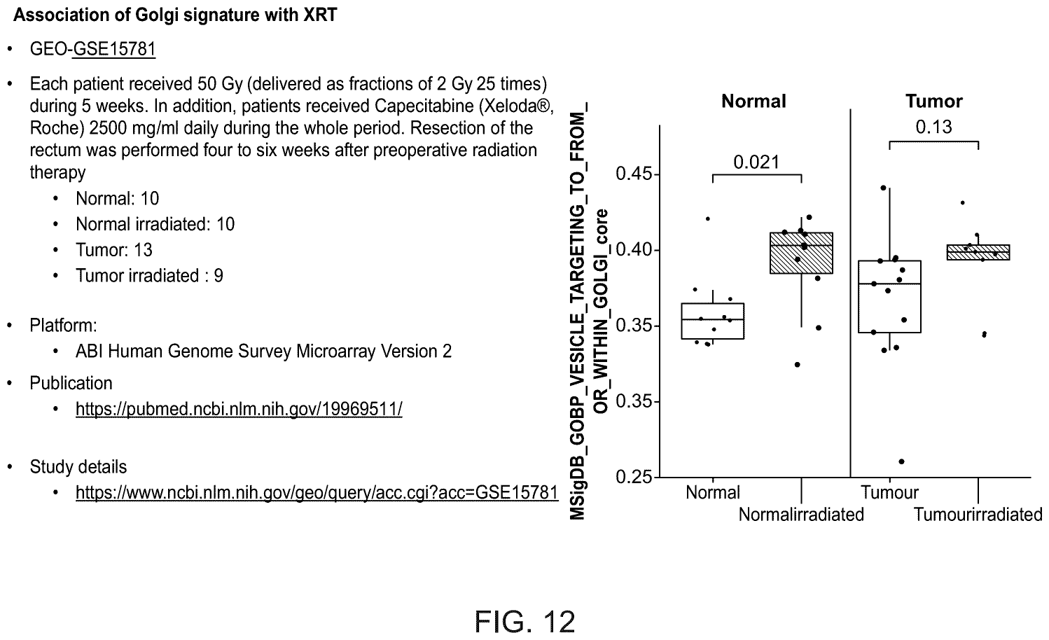

In order to assess the effect of XRT and SOC treatment on Golgi signature, we analyzed a public dataset in CRC which revealed that the signatures were enriched in radiated tissues (both normal and tumor) after 5 weeks of XRT treatment (FIG. 12) vs. separate controls. In the study, each patient received 50 Gy (delivered as fractions of 2 Gy 25 times) during 5 weeks. In addition, patients received Capecitabine (Xeloda®, Roche) 2500 mg/ml daily during the whole period. Resection of the rectum was performed four to six weeks after preoperative radiation therapy.

K. Identification of Potential “Partners” with Golgi Signatures to Extend the Prediction of Vidutolimod Response to Baseline “Hot” and to Increase the Overall Differentiation Between Responders and Non-Responders

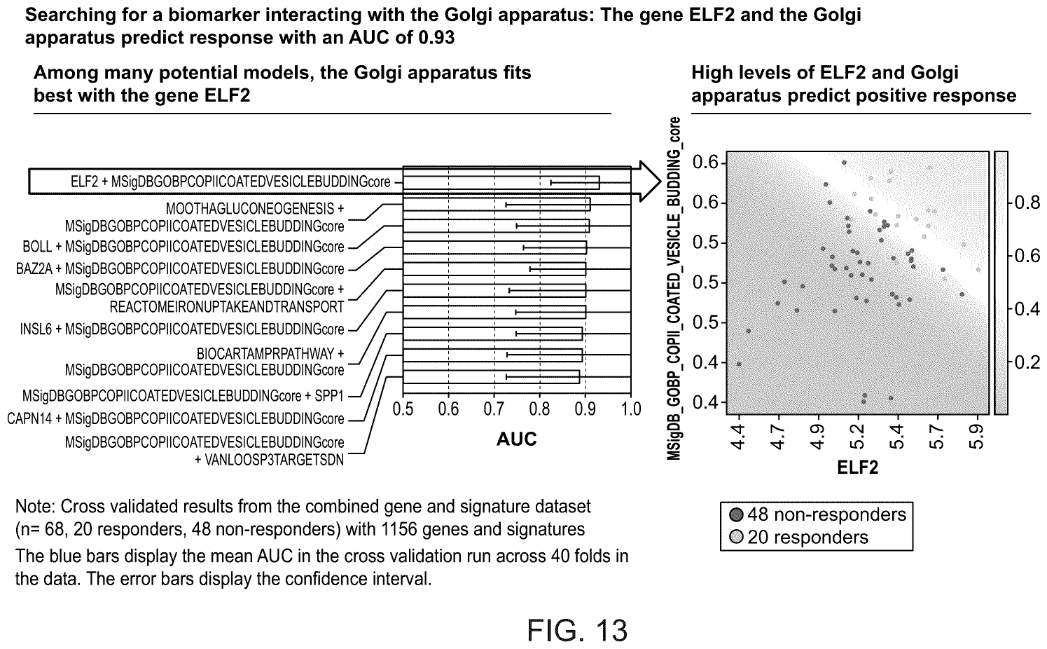

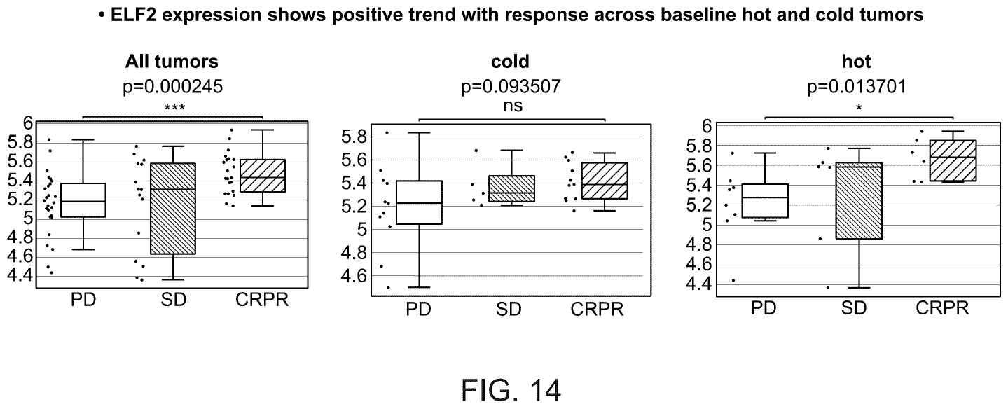

In order to identify one or more potential “partner” genes or signatures which potentially could be used in combination with the Golgi signature to predict treatment response in all baseline tumors from patients treated with vidutolimod, QLattice algorithm (from www.abzu.ai/) was applied. The best model with an AUC of 0.93 (95% CI: 0.82 to 1) is a gene “partner” ELF2, a transcription factor known to be important in immune regulation (FIG. 13). ELF2 expression shows a strong independent association with response across our dataset of PD-1 refractory melanoma baseline biopsies including hot and cold (FIG. 14). Other genes/signatures (e.g. MOOTHA_GLUCONEGENESIS, BOLL, BAZ2A, INSL6, etc.) were also identified that work with Golgi signature with slightly lower AUC.

L. Signature Correlation with Golgi Signature in Melanoma

In order to delineate the potential function of Golgi signatures in tumor microenvironment, all gene signatures were assessed for their correlation with Golgi signature in TCGA melanoma (SKCM). While those top (positive) correlated signatures are related to Vesicle-Golgi transporting (Table C), consistent with our expectations, the top anti-correlated signatures are more involved in immune cells or their states (Table D), including especially myeloid cells, such as macrophages, DC, and pDC. These data indicate that the presence of the Golgi signature may reflect a state of myeloid cell activation or function that is amenable to activation by vidutolimod. Further, these anti-correlated signatures may provide independent approaches to identifying novel gene signatures associated with response to innate immune activators such as vidutolimod in cancer immunotherapy.

TABLE C
Top 50 correlated signatures

| ID | correlations |
|---|---|
| MSigDB_GOBP_VESICLE_TARGETING_TO_FROM_OR_WITHIN_GOLGI_core | 1 |
| MSigDB_GOBP_COPII_VESICLE_core_com | 0.993 |
| MSigDB_GOBP_COPII_COATED_VESICLE_BUDDING_core | 0.99 |
| GO_COPII_COATED_VESICLE_BUDDING | 0.903 |
| GO_GOLGI_VESICLE_BUDDING | 0.9 |
| MSigDB_GOBP_COPII_COATED_VESICLE_BUDDING | 0.896 |
| GO_VESICLE_TARGETING_TO_FROM_OR_WITHIN_GOLGI | 0.886 |
| MSigDB_GOBP_VESICLE_TARGETING_TO_FROM_OR_WITHIN_GOLGI | 0.881 |
| GO_VESICLE_TARGETING | 0.872 |
| GO_VESICLE_BUDDING_FROM_MEMBRANE | 0.858 |
| REACTOME_COPII_MEDIATED_VESICLE_TRANSPORT | 0.85 |
| GO_ENDOPLASMIC_RETICULUM_TO_GOLGI_VESICLE_MEDIATED_TRANSPORT | 0.778 |
| REACTOME_CARGO_CONCENTRATION_IN_THE_ER | 0.757 |
| MSigDB_HALLMARK_PROTEIN_SECRETION_core | 0.727 |
| TIEN_INTESTINE_PROBIOTICS_24HR_DN | 0.727 |
| SEIDEN_ONCOGENESIS_BY_MET | 0.722 |
| SENESE_HDAC3_TARGETS_UP | 0.718 |
| GSE1460_DP_THYMOCYTE_VS_THYMIC_STROMAL_CELL_DN | 0.717 |
| xCell_Preadipocytes_ENCODE_3 | 0.716 |
| JI_RESPONSE_TO_FSH_DN | 0.714 |
| BCAT_BILD_ET_AL_DN | 0.713 |
| DITTMER_PTHLH_TARGETS_UP | 0.712 |
| SENESE_HDAC1_TARGETS_UP | 0.712 |
| REACTOME_SIGNALING_BY_EGFR | 0.704 |
| GO_GOLGI_VESICLE_TRANSPORT | 0.703 |
| WANG_CLIM2_TARGETS_DN | 0.699 |
| TIEN_INTESTINE_PROBIOTICS_2HR_DN | 0.697 |
| BLUM_RESPONSE_TO_SALIRASIB_UP | 0.697 |
| JOHNSTONE_PARVB_TARGETS_1_DN | 0.697 |
| MSigDB_HALLMARK_PROTEIN_SECRETION | 0.696 |
| TBK1.DF_DN | 0.696 |
| SENESE_HDAC2_TARGETS_UP | 0.692 |
| REACTOME_SIGNALING_BY_EGFR_IN_CANCER | 0.691 |
| PGF_UP.V1_UP | 0.69 |
| HALLMARK_PROTEIN_SECRETION | 0.69 |
| GSE37301_GRANULOCYTE_MONOCYTE_PROGENITOR_VS_RAG2_KO_NK_CELL_UP | 0.688 |
| GSE1460_NAIVE_CD4_TCELL_CORD_BLOOD_VS_THYMIC_STROMAL_CELL_DN | 0.687 |
| GO_ER_NUCLEUS_SIGNALING_PATHWAY | 0.686 |
| GSE42021_TCONV_PLN_VS_CD24INT_TCONV_THYMUS_UP | 0.685 |
| PUIFFE_INVASION_INHIBITED_BY_ASCITES_DN | 0.683 |
| MSigDB_DITTMER_PTHLH_TARGETS_DN_core | 0.683 |
| IKEDA_MIR1_TARGETS_UP | 0.678 |
| ACTTTAT_MIR1425P | 0.678 |
| GSE20198_UNTREATED_VS_IL12_TREATED_ACT_CD4_TCELL_UP | 0.677 |
| DE_YY1_TARGETS_DN | 0.676 |
| CSR_EARLY_UP.V1_UP | 0.676 |
| ENK_UV_RESPONSE_KERATINOCYTE_DN | 0.675 |
| WELCSH_BRCA1_TARGETS_UP | 0.673 |
| BILD_SRC_ONCOGENIC_SIGNATURE | 0.672 |
| SMITH_TERT_TARGETS_UP | 0.672 |

TABLE D
Top 50 anti-correlated signatures

| ID | correlations |
|---|---|
| GSE16385_MONOCYTE_VS_12H_IL4_TREATED_MACROPHAGE_DN | −0.585910662 |
| BONOME_OVARIAN_CANCER_POOR_SURVIVAL_DN | −0.585200004 |
| GSE45365_WT_VS_IFNAR_KO_CD11B_DC_UP | −0.579057624 |
| GSE2405_0H_VS_1.SH_A_PHAGOCYTOPHILUM_STIM_NEUTROPHIL_DN | −0.578321851 |
| GSE9006_TYPE_1_VS_TYPE_2_DIABETES_PBMC_AT_DX_UP | −0.562108318 |
| GSE45837_WT_VS_GFI1_KO_PDC_DN | −0.545841411 |
| GINESTIER_BREAST_CANCER_ZNF217_AMPLIFIED_DN | −0.539465907 |
| MORF_IKBKG | −0.536586702 |
| GSE37532_VISCERAL_ADIPOSE_TISSUE_VS_LN_DERIVED_PPARG_KO_TREG_CD4_TCELL_DN | −0.531197499 |
| SRC_UP.V1_UP | −0.526930305 |
| GSE38681_WT_VS_LYL1_KO_LYMPHOID_PRIMED_MULTIPOTENT_PROGENITOR_UP | 0.522877124 |
| GSE16266_CTRL_VS_HEATSHOCK_AND_LPS_STIM_MEF_DN | −0.513845513 |
| FAELT_B_CLL_WITH_VH3_21_UP | −0.513539428 |
| GSE9006_HEALTHY_VS_TYPE_2_DIABETES_PBMC_AT_DX_UP | −0.5066297 |
| MORF_ARAF1 | −0.504451571 |
| GSE22103_UNSTIM_VS_GMCSF_AND_IFNG_STIM_NEUTROPHIL_DN | −0.498713181 |
| GSE36392_TYPE_2_MYELOID_VS_MAC_IL25_TREATED_LUNG_DN | −0.494305459 |
| MORF_FDXR | −0.491970263 |
| GSE19401_RETINOIC_ACID_VS_RETINOIC_ACID_AND_PAM2CSK4_STIM_FOLLICULAR_DC_DN | −0.487620117 |
| GSE43863_NAIVE_VS_LY6C_LOW_CXCR5NEG_CD4_EFF_TCELL_D6_LCMV_DN | −0.48549331 |
| GSE21678_WT_VS_FOXO1_FOXO3_KO_TREG_DN | −0.48477726 |
| GSE [symbol] 099_UNSTIM_VS_MCSF_TREATED_MONOCYTE_DAY7_DN | −0.483672738 |
| GO_ADENOSINE_DEAMINASE_ACTIVITY | −0.482088237 |
| GO_GTPASE_ACTIVATING_PROTEIN_BINDING | −0.480150119 |
| GSE42021_CD24HI_TREG_VS_CD24HI_TCONV_THYMUS_UP | −0.476978854 |
| MORF_DDX11 | −0.476780483 |
| GSE21 [symbol] 60_NAIVE_VS_TERTIARY_MEMORY_CD8_TCELL_DN | −0.47535452 |
| MORF_PDPK1 | −0.473193674 |
| GSE36891_UNSTIM_VS_PAM_TLR2_STIM_PERITONEAL_MACROPHAGE_DN | −0.472866054 |
| MORF_CNTN1 | −0.472190855 |
| GSE23114_WT_VS_SLE2C1_MOUSE_SPLEEN_B1A_BCELL_DN | −0.470952526 |
| SANSOM_APC_TARGETS_REQUIRE_MYC | −0.467044337 |
| GSE4 [symbol] 84_LPS_VS_VEHICLE_CTRL_TREATED_DC_UP | −0.46686483 |
| GSE2405_S_AUREUS_VS_A_PHAGOCYTOPHILUM_NEUTROPHIL_DN | −0.465619348 |
| GSE6259_BCELL_VS_CD4_TCELL_UP | −0.463860184 |
| GSE42724_NAIVE_VS_B1_BCELL_DN | −0.463444327 |
| MODULE_132 | −0.461618545 |
| KUUSELO_PANCREATIC_CANCER_19Q13_AMPLIFICATION | −0.461274267 |
| GSE22589_HEALTHY_VS_SIV_INFECTED_DC_UP | −0.459521945 |
| GSE4 [symbol] 365_HEALTHY_VS_MCMV_INFECTION_CD8A_DC_IFNAR_KO_UP | −0.458716241 |
| MORF_PTPN9 | −0.458194353 |
| LU_EZH2_TARGETS_UP | −0.456566558 |
| GSE5 [symbol] 80_UNSTIM_VS_45MIN_LPS_STIM_MACROPHAGE_DN | −0.455535536 |
| GSE45365_WT_VS_IFNAR_KO_CD11B_DC_MCMV_INFECTION_UP | −0.454523072 |
| GSE37301_MULTIPOTENT_PROGENITOR_VS_COMMON_LYMPHOID_PROGENITOR_UP | −0.454427605 |
| GSE41176_UNSTIM_VS_ANTI_IGM_STIM_TAK1_KO_BCELL_24H_UP | −0.453692967 |
| MODULE_101 | −0.452845967 |
| GO_DEOXYRIBOSE_PHOSPHATE_CATABOLIC_PROCESS | −0.452262372 |
| MORF_CASP2 | −0.451307664 |
| GSE32423_IL7_VS_IL7_IL4_MEMORY_CD8_TCELL_DN | −0.451163727 |

[symbol]

indicates data missing or illegible when filed

M. Association of Golgi Signature with BRAF Mutation Status and Baseline Risk Factors

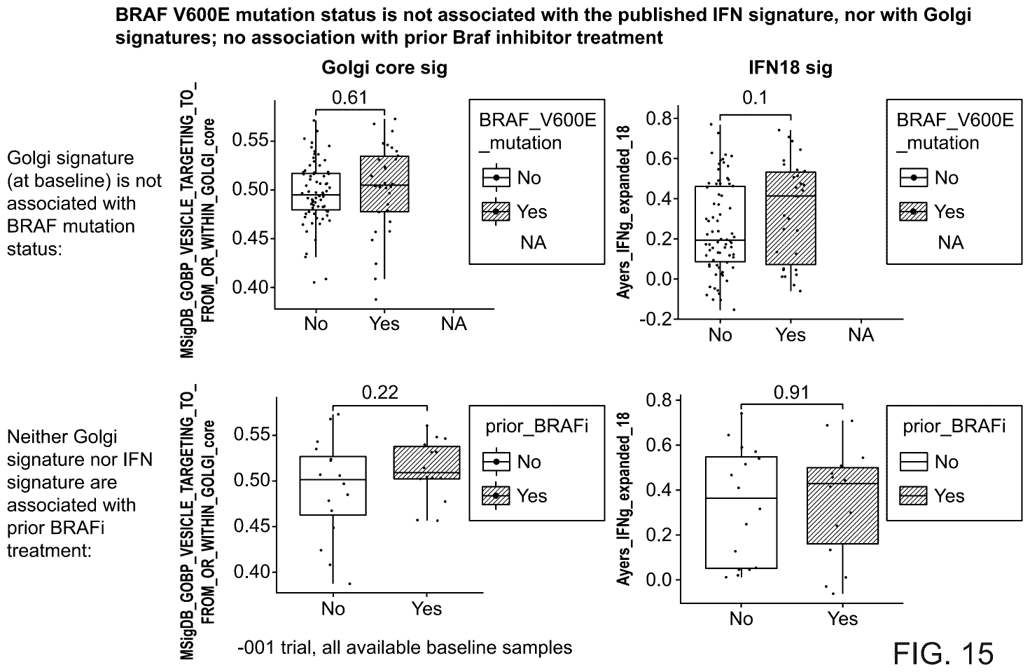

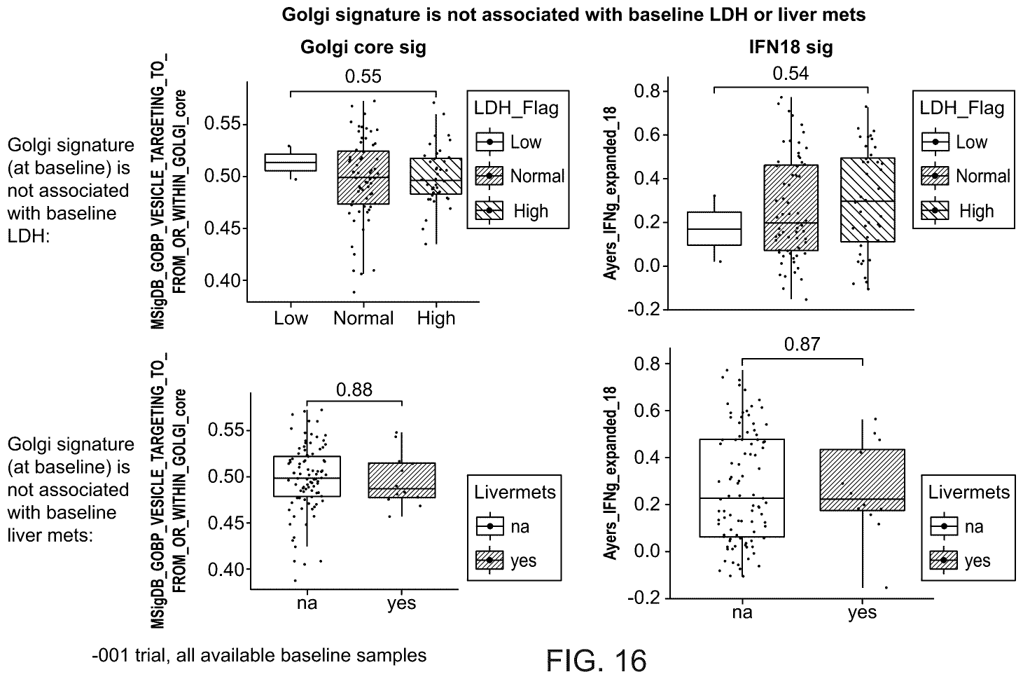

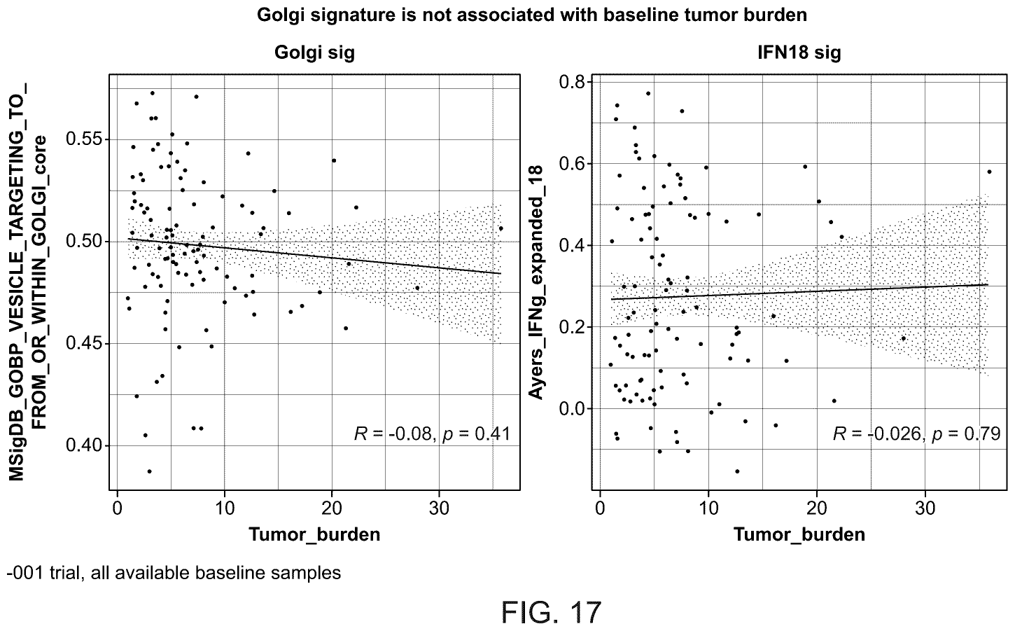

In the PD-1 refractory melanoma dataset the Golgi signatures expression showed no association with risk factors such as BRAF mutation status or prior BRAFi/MEKi treatment (FIG. 15); baseline LDH level or Liver mets (FIG. 16), or baseline tumor burden (FIG. 17), providing further evidence that the Golgi signature expression is not a surrogate for other known prognostic factors.

N. High Baseline Macrophage/Monocytes or T_{reg} are Independent Predictors of Nonresponse or PD to Vidutolimod Combination with Pembrolizumab

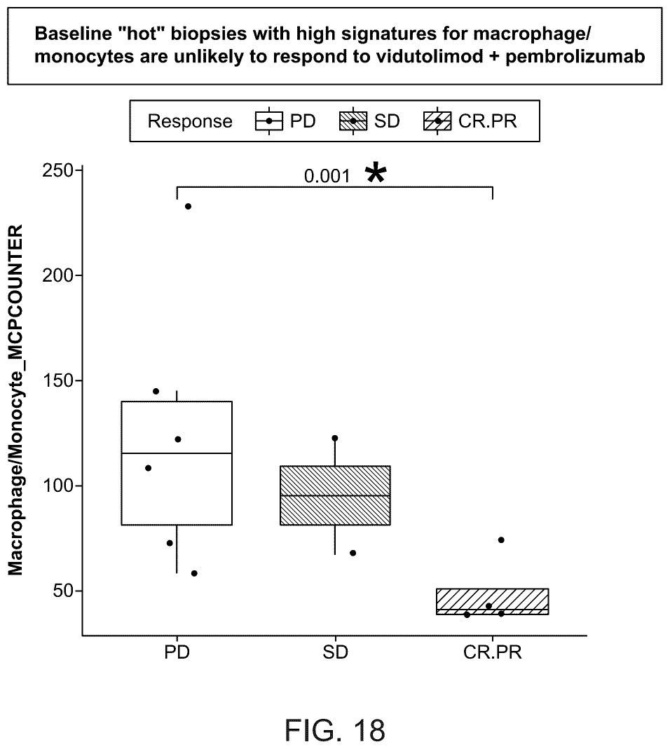

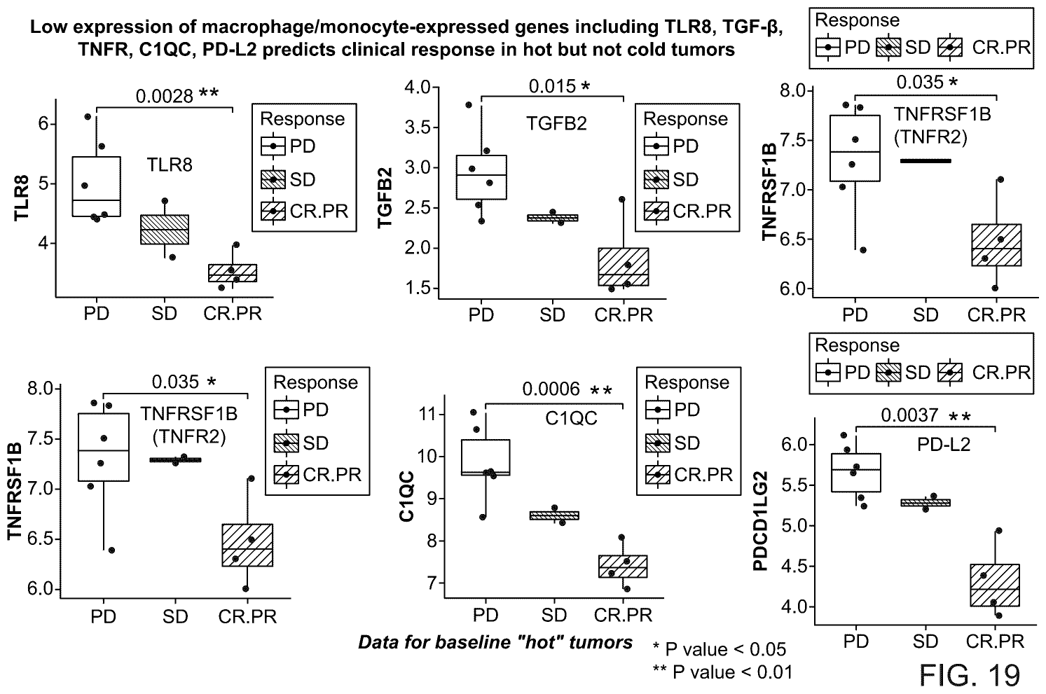

Macrophage/monocyte signatures were found to be associated with PD, especially in baseline “hot” tumors from CMP-001-001 data (FIG. 18). High myeloid cells in tumors are known to be associated with resistance to immunotherapies. Signatures or other indicators of the presence of high myeloid cells in baseline tumor biopsies, in some embodiments, may be a component of a signature that can be used for patient selection for vidutolimod therapy, alone or in concert with a Golgi vidutolimod response signature. For example, individual genes that are expressed in macrophages and/or monocytes and show strong inverse associations with response to vidutolimod, especially in baseline hot tumors, include TLR8, TGFB2, TNFRSF1A, TNFRSF1B, C1QC, and PD-L1 (FIG. 19). The RNA levels of one or more of these genes can comprise a part of a biomarker for this purpose, where relatively low levels of expression of these genes indicates a low level of immune suppressive macrophages or monocytes and whereby patients with relatively low levels of expression of these genes are predicted to have a higher probability of response to an innate immune activator compared to patients with higher levels of expression of these genes in their baseline tumor biopsies. As used herein, “detecting the presence of a low macrophage and/or monocyte cell population” means detecting either a relatively low macrophage signature such as Macrophage/Monocyte_MCPCOUNTER or a relatively low level of expression of one or more of the myeloid-associated genes TLR8, TGFB2, TNFRSF1A, TNFRSF1B, C1QC, and PD-L1.

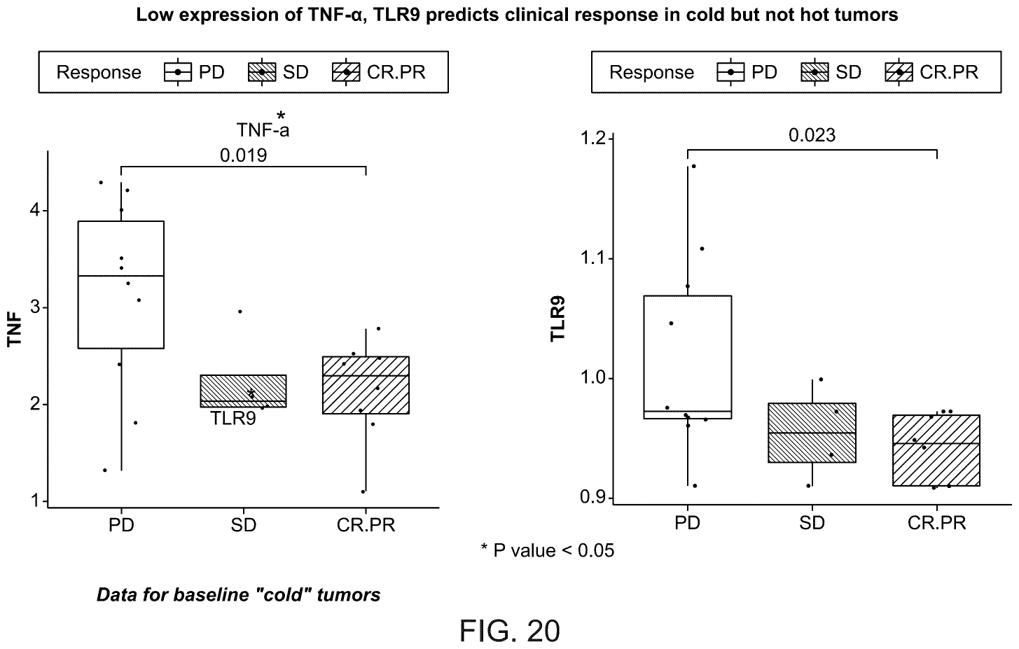

Conversely in baseline “cold” tumors but not in hot tumors, low expression of TLR9 and TNFA are associated with a higher probability of response to an innate immune activator compared to patients with higher levels of expression of these genes in their baseline tumor biopsies (FIG. 20).

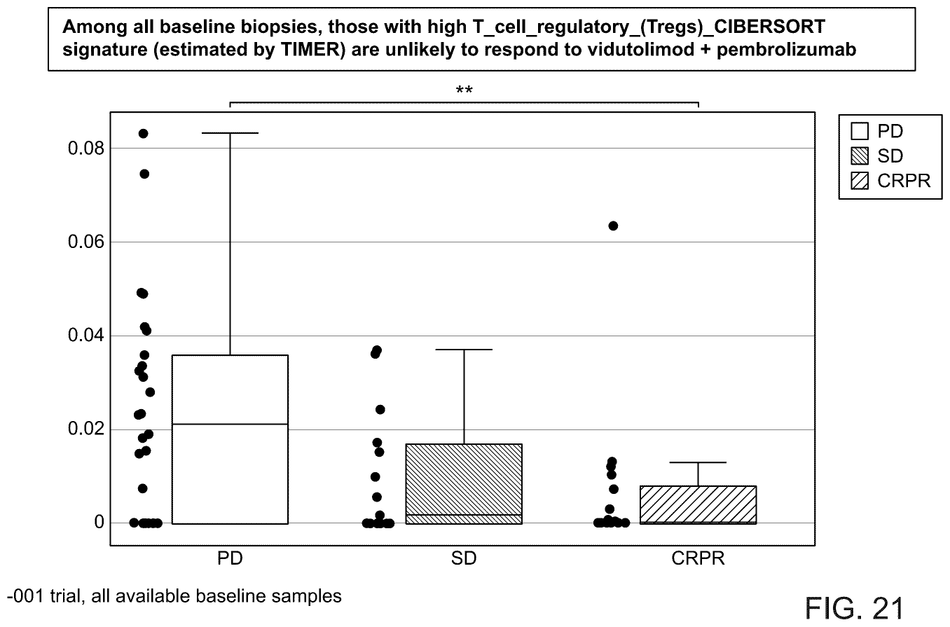

In addition, high expression of a T regulatory (T_{reg}) signature was found to be associated with PD in all baseline samples (FIG. 21). Signatures or other indicators of the presence of high T_{reg} cells in baseline tumor biopsies, in some embodiments, may be a component of a signature that can be used for patient selection for vidutolimod therapy, alone or in concert with a Golgi vidutolimod response signature.

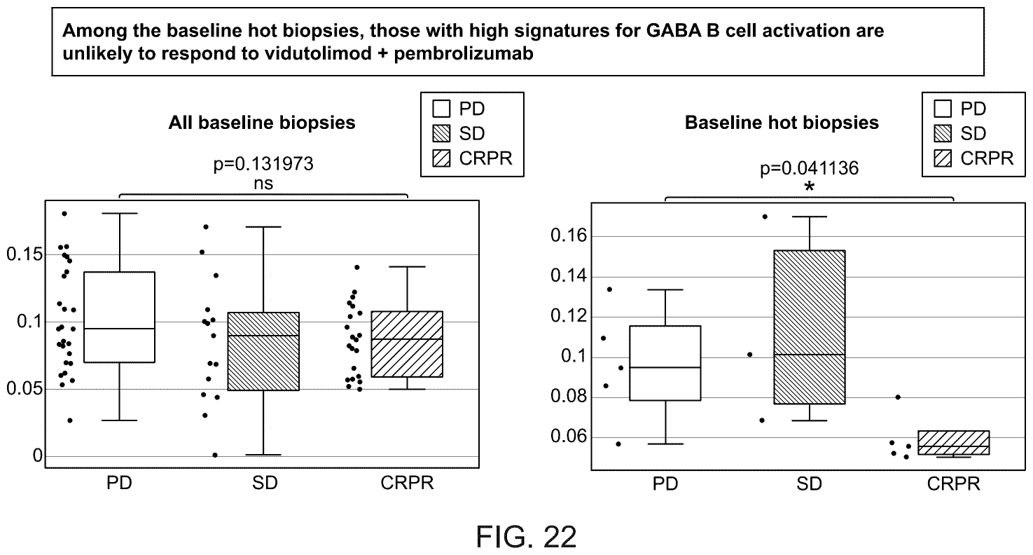

Finally, a signature related to GABA-producing B cells (REACTOME_GABA_B_RECEPTOR_ACTIVATION) was found to be associated with PD in baseline “hot” tumors (FIG. 22). Signatures or other indicators of the presence of high GABA-producing B cells in baseline tumor biopsies, in some embodiments, may be a component of a signature that can be used for patient selection for vidutolimod therapy, alone or in concert with a Golgi vidutolimod response signature.

To summarize, the present Examples collectively demonstrate for vidutolimod, and by extension, to other TLR9 agonists, other TLR agonists, and other innate immune activators:
1. High expression of some or all of the genes within the Golgi vidutolimod response signature is an independent predictor for response to vidutolimod, especially in patients whose baseline biopsies are not “hot.”
2. High expression of ELF2 is an independent predictor for response to vidutolimod, and may be used in combination with one or more of the Golgi signatures to provide more accurate prediction of the probability of response to vidutolimod alone or in combination with other immunotherapies such as PD-1 blockade.
3. Low expression of signatures or genes associated with T_{reg}, and/or macrophage/monocytes are independent predictors for response to vidutolimod+/−PD-1 blockade, and may be used in combination with one or more of the Golgi signatures to provide more accurate prediction of the probability of response to vidutolimod alone or in combination with other immunotherapies such as PD-1 blockade.
4. Low expression of a B cell GABA signature is an independent predictor for response to vidutolimod+/−PD-1 blockade in patients with baseline “hot” tumors, and may be used in combination with one or more of the Golgi signatures to provide more accurate prediction of the probability of response to vidutolimod alone or in combination with other immunotherapies such as PD-1 blockade.

1. A method of identifying a subject that is likely to benefit from a cancer immunotherapy comprising an innate immune activator, said method comprising:
(i) obtaining a tumor sample from a subject with cancer; and
(ii) detecting the expression level in the sample of at least 5 vidutolimod response core signature gene transcription products selected from the group consisting of SEC16B, AREG, COL7A1, SEC24D, CNIH2, MAPK15, TGFA, CUL3, SAR1B, USO1, TRAPPC6A, SEC24A, TMED2, SEC31A, BET1, GOLGA2, RAB1A, NAPA, LMAN1, TRAPPC3, PREB, SCFD1, SEC23A, CTSC, TRAPPC1, ANKRD28, SEC24B, MCFD2, CD59, TMED10, STX5, TRAPPC6B, PPP6R3, SEC13, TRAPPC9, CEP19, ARFGAP3, GOSR1, SEC23B, MIA3, and VAPA;
wherein said detecting of an enriched level of expression identifies a subject that is likely to benefit from a cancer immunotherapy comprising an innate immune activator.

2. The method of claim 1, further comprising a step of confirming that the tumor is not “hot”:
wherein said detecting of an enriched level of expression of at least 5 vidutolimod core signature gene transcription products in a tumor that is not “hot” identifies a subject that is likely to benefit from a cancer immunotherapy comprising an innate immune activator.

3. The method of claim 1, further comprising a step of detecting the presence of a low macrophage and/or monocyte cell population; wherein said detecting identifies a subject that is likely to benefit from a cancer immunotherapy comprising an innate immune activator.

4. The method of claim 1, further comprising a step of detecting one or more of the following in the tumor sample:
(a) the elevated expression of ELF2; and/or
(b) the presence of a low T_{reg} cell population; and/or
(c) low expression of a GABA B cell signature; and/or
(d) the presence of a low macrophage and/or monocyte cell population;
wherein said detecting identifies a subject that is likely to benefit from a cancer immunotherapy comprising an innate immune activator.

5. (canceled)

6. A method of treating a subject afflicted with cancer with a cancer immunotherapy comprising an innate immune activator, said method comprising:
(i) obtaining a tumor sample from a subject with cancer; and
(ii) detecting the expression level in the sample of at least 5 vidutolimod response core signature gene transcription products selected from the group consisting of SEC16B, AREG, COL7A1, SEC24D, CNIH2, MAPK15, TGFA, CUL3, SAR1B, USO1, TRAPPC6A, SEC24A, TMED2, SEC31A, BET1, GOLGA2, RAB1A, NAPA, LMAN1, TRAPPC3, PREB, SCFD1, SEC23A, CTSC, TRAPPC1, ANKRD28, SEC24B, MCFD2, CD59, TMED10, STX5, TRAPPC6B, PPP6R3, SEC13, TRAPPC9, CEP19, ARFGAP3, GOSR1, SEC23B, MIA3, and VAPA, and, where an enriched level of expression is detected,
(iii) administering a cancer immunotherapy comprising an innate immune activator to the subject.

7. The method of claim 6, further comprising a step of confirming that the tumor is not “hot”.

8. The method of claim 6, further comprising a step of detecting the presence of a low macrophage and/or monocyte cell population; wherein the elevated expression and/or detection of low macrophage and/or monocyte population identifies a subject that is likely to benefit from said cancer immunotherapy comprising an innate immune activator.

9. The method of claim 6, further comprising a step of:
detecting one or more of the following in the tumor sample:
(a) the elevated expression of ELF2; and/or
(b) the presence of a low T_{reg} cell population; and/or
(c) low expression of a GABA B cell signature; and/or
(d) the presence of a low macrophage and/or monocyte cell population.

10-11. (canceled)

12. The method of claim 1, wherein the innate immune activator is a TLR agonist,
wherein the TLR agonist is selected from the group consisting of a TLR9, TLR4, TLR7, and TLR8 agonist.

13. The method of claim 12, wherein the TLR agonist is a TLR9 agonist,
optionally wherein the TLR9 agonist is selected from the group consisting of A-class CpG DNA, C-class CpG DNA, E-class CpG DNA, A/E-class CpG DNA, P-class CpG DNA, and any combination thereof.

14. (canceled)

15. The method of claim 13, wherein the TLR9 agonist is an A-class CpG DNA,
optionally wherein the A-class CpG DNA comprises the sequence
(SEQ ID NO: 1)
GGGGGGGGGGGACGATCGTCGGGGGGGGGG

16. (canceled)

17. The method of claim 15, wherein the A-class CpG DNA is formulated as a virus-like particle (VLP),
optionally wherein the VLP comprises bacteriophage Qβ coat protein.

18. (canceled)

19. The method of claim 1, wherein the tumor is determined to be not “hot” on the basis of one or more of the following:
(a) a TIDE score <−1;
(b) low expression of an IFN-γ transcription signature comprising 5 or more of CD3D, IDO1, CIITA, CD3E, CCL5, GZMK, CD2, HLA-DRA, CXCL13, IL2RG, NKG7, HLA-E, LAG3, TAGAP, CXCL10, STAT1, GZMB, CXCR6 gene transcription products;
(c) a low Immunoscore; and/or
(d) a PD-L1 CPS<10.

20. The method of claim 1, wherein the tumor is refractory to an inhibitor of a checkpoint selected from the group consisting of PD-1 and PD-L1.

21. The method of claim 1, wherein the subject is a human afflicted with one or more cancerous tumors,
optionally wherein the cancerous tumor is a lymphoma or a cancerous tumor of a tissue or organ selected from the group consisting of skin, head and neck, esophagus, stomach, liver, colon, rectum, pancreas, lung, breast, cervix, ovary, kidney, bladder, prostate, thyroid, brain, muscle, and bone,
optionally wherein the cancerous tumor is melanoma, NSCLC, and HNSCC
optionally wherein the subject has received a therapy selected from the group consisting of radiotherapy, chemotherapy, immunotherapy, a therapy comprising a checkpoint inhibitor, surgery, hormone therapy.

22-24. (canceled)

25. The method of claim 1, wherein the detecting of a gene transcription product is selected from the group of techniques consisting of RNA sequencing (RNA-Seq), mRNA sequencing (mRNA-Seq), targeted RNA-Seq, and noncoding RNA-Seq, Nanostring, microarrays, or other hybridization based techniques.

26. The method of claim 1, wherein the presence of 10, 15, 20, 25, 30, 35 or more vidutolimod core signature gene transcription products are detected,
wherein the detected vidutolimod core signature gene transcription products have an enrichment score in the highest 80% of patient biopsies for the tumor type,
wherein the detected vidutolimod core signature gene transcription products are expressed in one or more cells selected from the group consisting of plasmacytoid dendritic cells (pDC), plasma cells, mast cells, activated monocytes, macrophages, dendritic cells, and/or T cells.

27-28. (canceled)

29. The method of claim 6, wherein the cancer immunotherapy is administered via intratumoral, peritumoral, systemic, intravenous, intraperitoneal, enteric, oral, intramuscular, subcutaneous, transmucosal, topical, intravesicular and/or transdermal routes.

30. (canceled)

31. The method of claim 6, further comprising administering to the subject at least one dose of a composition comprising a checkpoint inhibitor (CPI),
optionally wherein the CPI is an antibody or antigen-binding fragment thereof which binds specifically to an antigen selected from the group consisting of PD-1, PD-L1, LAG-3, TIM-3, and CTLA-4.

32-34. (canceled)

35. A kit comprising one or more compositions comprising reagents capable of detecting
at least 5 vidutolimod core signature gene transcription products selected from the group consisting of SEC16B, AREG, COL7A1, SEC24D, CNIH2, MAPK15, TGFA, CUL3, SAR1B, USO1, TRAPPC6A, SEC24A, TMED2, SEC31A, BET1, GOLGA2, RAB1A, NAPA, LMAN1, TRAPPC3, PREB, SCFD1, SEC23A, CTSC, TRAPPC1, ANKRD28, SEC24B, MCFD2, CD59, TMED10, STX5, TRAPPC6B, PPP6R3, SEC13, TRAPPC9, CEP19, ARFGAP3, GOSR1, SEC23B, MIA3, and VAPA; or
one or more of:
(a) the presence of a low macrophage and/or monocyte cell population;
(b) high expression of ELF2; and/or
(c) the presence of a low T_{reg} cell population; and/or
(d) low expression of a GABA B cell signature.

36. (canceled)
